(19) United States
(12) Patent Application Publication
(10) Pub. No.: US 2014/0200333 A1
(43) Pub. Date: Jul. 17, 2014

(54) DENDRON REPORTER MOLECULES

(71) Applicants: Ewald Terpetschnig, Urbana, IL (US); Inna G. Yermolenko, Kharkov (UA); Olena M. Obukhova, Kharkov (UA); Anatoliy Tatarets, Kharkov (UA); Leonid D. Patsenker, Kharkov (UA)

(72) Inventors: Ewald Terpetschnig, Urbana, IL (US); Inna G. Yermolenko, Kharkov (UA); Olena M. Obukhova, Kharkov (UA); Anatoliy Tatarets, Kharkov (UA); Leonid D. Patsenker, Kharkov (UA)

(73) Assignee: SETA BIOMEDICALS, LLC, Urbana, IL (US)

(21) Appl. No.: 14/238,829

(22) PCT Filed: Aug. 14, 2012
(86) PCT No.: PCT/US2012/050827

§ 371 Date: Feb. 13, 2014

Related U.S. Application Data
(60) Provisional application No. 61/523,674, filed on Aug. 15, 2011.

(51) Int. Cl. C07D 471/06; C07D 403/14

(52) U.S. Cl. CPC C07D 471/06; C07D 403/14
USPC 530/391.5; 548/455; 548/255; 544/245

(57) ABSTRACT

Dendronic reporters are described which incorporate a high density of luminescent or non-luminescent dyes at periphery sites and a focal point group that is reactive, ionic or a conjugated substance. Such dendronic reporters are capable of sensing analytes, or are otherwise useful in luminescent assays. Additionally, methods of synthesis are described.

### RELATED APPLICATION

This application claims the benefit of U.S. provisional application No. 61/523,674, filed 15 Aug. 2011, the entire disclosure of which is incorporated herein by reference.

### FIELD OF THE INVENTION

The invention relates to dendron-based reporter compounds. More particularly, the invention relates to reporter compounds that allow to overcome some of the shortcomings of conventional reporters such as low sensitivity, low extinction coefficients, low photostability among others.

### BACKGROUND

Colorimetric and/or luminescent compounds may offer researchers the opportunity to use color and light to analyze samples, investigate reactions, and perform assays, either qualitatively or quantitatively. Generally, brighter, more photostable reporters may permit faster, more sensitive, and more selective methods to be utilized in such research.

While a colorimetric compound absorbs light, and may be detected by that absorbance, a luminescent compound, or luminophore, is a compound that emits light. A luminescence method, in turn, is a method that involves detecting light emitted by a luminophore, and using properties of that light to understand properties of the luminophore and its environment. Luminescence methods may be based on chemiluminescence and/or photoluminescence, among others, and may be used in spectroscopy, microscopy, immunoassays, and hybridization assays, among others.

A chromophore is a part of a molecule responsible for the light absorption. It may also be a fluorophore. A fluorescent or luminescent reporter (fluorophore or luminophore) is a molecule or a part of a molecule that provides a fluorescence or luminescence signal that is of sufficient character to be detected. In this disclosure, a luminophore and fluorophore are used interchangeably. A dye is a compound that absorbs light in the ultraviolet (UV), visible, near-infrared (NIR, near-IR), or infrared (IR) spectral range. It may be a fluorescent (luminescent) reporter or a quencher of fluorescence (luminescence). A quencher of fluorescence (luminescence) is a molecule or a part of a molecule, the fluorescence (luminescence) of which is not strong enough to be measured and/or that reduces fluorescence (luminescence) quantum yield of a fluorophore. Quenchers can be used as reporters in photophysical measurements. An environment-sensitive molecule or compound is a molecule or compound where the spectral or other photophysical characteristics of which depend on its microenvironment. The environment-sensitive molecules include, but are not limited to, pH-sensitive, polarity sensitive and potential sensitive molecules, and ion indicators.

Photoluminescence is a particular type of luminescence that involves the absorption and subsequent re-emission of light. In photoluminescence, a luminophore is excited from a low-energy ground state into a higher-energy excited state by the absorption of a photon of light. The energy associated with this transition is subsequently lost through one of several mechanisms, including production of a photon through fluorescence or phosphorescence.

Photoluminescence may be characterized by a number of parameters, including extinction coefficient, excitation and emission spectrum, Stokes' shift, luminescence lifetime, and quantum yield. The extinction coefficient is a wavelength-dependent measure of the absorbing power of a luminophore. The excitation spectrum is the dependence of emission intensity upon the excitation wavelength, measured at a single constant emission wavelength. The emission spectrum is the wavelength distribution of the emission, measured after excitation with a single constant excitation wavelength. The Stokes' shift is the difference in wavelengths between the maximum of the emission spectrum and the maximum of the absorption spectrum. The luminescence lifetime is the average time that a luminophore spends in the excited state prior to returning to the ground state and emitting a photon. The quantum yield is the ratio of the number of photons emitted to the number of photons absorbed by a luminophore. The brightness, a wavelength-dependent measure, is the product of the quantum yield and the extinction coefficient.

Luminescence methods may be influenced by extinction coefficient, excitation and emission spectra, Stokes' shift, and quantum yield, among others, and may involve characterizing fluorescence intensity, fluorescence polarization (FP), fluorescence resonance energy transfer (FRET), fluorescence lifetime (FLT), total internal reflection fluorescence (TIRF), fluorescence correlation spectroscopy (FCS), fluorescence recovery after photobleaching (FRAP), and their phosphorescence analogs, among others.

Luminescence methods have several significant potential strengths. First, luminescence methods may be very sensitive, because modern detectors, such as photomultiplier tubes (PMTS) and charge-coupled devices (CCDs), can detect very low levels of light. Second, luminescence methods may be very selective, because the luminescence signal may come almost exclusively from the luminophore.

Despite these potential strengths, luminescence methods may suffer from a number of shortcomings, at least some of which relate to the luminophore. For example, the luminophore may have an extinction coefficient, quantum yield or brightness that is too low to permit detection of an adequate amount of light. The luminophore also may have a Stokes' shift that is too small to permit effective detection of emission light without significant detection of excitation light. The luminophore also may have an excitation spectrum that does not permit it to be excited by wavelength-limited light sources, such as common lasers and arc lamps. The luminophore also may be unstable, so that it is readily bleached and rendered non-luminescent. The luminescent compound may not be able to passively cross the plasma membrane in cells due to the presence of one or more ionic charges. The luminophore also may have an excitation or emission spectrum that overlaps with the well-known auto-luminescence of biological and other samples; such auto-luminescence is particularly significant at wavelengths below about 600 nm and typically dominant at wavelengths below 400 nm. The luminophore also may be expensive, especially if it is difficult to manufacture.

One of the main issues related to organic labels is that they exhibit severe quenching upon labeling to proteins and other complex biomolecules at higher dye to protein ratios (D/P, the number of dye groups per protein). For example, the quantum yield of Cy5 is reduced on average by over 70% upon increasing the D/P from 1 to 5 on an antibody.

Because of these shortcomings the use of conventional dye reporters are limited. Thus there exists the need to provide improved optical labels which strongly absorb, reduce the possibility of biological interference, and, for luminescent labels, brightly emit.

Dyes may also be bound to dendritic or dendronic macromolecules to provide optical labels. The dendritic or dendronic macromolecule provides a structured, light weight backbone to host a dye and reactive groups.

Dendrimers are repetitively branched macromolecules that form a core-shell structure (Astruc et at. (2010), Chem. Rev. 110 (4): 1857-1959). Dendrimers consist of two or more multivalent, branched units (dendron units) emanating from a single central atom, atomic cluster or molecular structure called the core. They are comprised of repeated radial layers of dendron units in a precise pattern (layer upon layer). Each layer approximately concentrically covers the prior layer and forms a generation of the dendrimer. The number of layers is the number of generations of the dendrimer. The final layer forms a periphery and exposed dendron unit sites along the periphery are available to host reactive groups, ionic groups or other conjugated substances. Dendrimers may consist of uniform or non-uniform dendron units and may be symmetric or asymmetric. However, all dendrimers retain the core-shell structure.

Dendrons are much like dendrimers except that all branches emanate from single atom, atomic cluster or molecular structure called a focal point (http://en.wikipedia.org/wiki/Dendrimer; Nanjwade, Basavaraj K.; Hiren M. Bechraa, Ganesh K. Derkara, F. V. Manvia, Veerendra K. Nanjwade (2009), “Dendrimers: Emerging polymers for drug-delivery systems”. European Journal of Pharmaceutical Sciences 38 (3): 185-196). Such structures form a molecular tree rather than the core-shell of a dendrimer. Dendrons consist of one or more multivalent, branched units (dendron units). The units repeat and form radial, approximately concentric layers, called generations, much like dendrimers. The terminal layer is called the periphery and all dendron unit sites not connected to the interior structure are available to host reactive groups, ionic groups or conjugated substances. Unlike dendrimers, the focal point remains available to host its own reactive groups, ionic groups or conjugated substances.

### SUMMARY OF THE INVENTION

This invention relates generally to functionalized dendronic reporters that combine as many dye components in a small volume element as possible, in addition to at least one reactive or ionic group for labeling to various molecules or carriers including a protein, a lectin, a nucleotide, an oligonucleotide, a peptide and a polypeptide, a particle, a nanoparticle, a protein nucleic acid, a phospholipid, an amino acid, a nucleic acid, a protein nucleic acid, a sugar, a polysaccaride, an oligosaccharide, a metallic nanoparticle, a quantum dot, a cell, a solid surface, a second fluorescent or non-fluorescent dye, a small drug and tyramide for use in, but not limited to, biological applications. The invention also relates to functionalized dendronic reporters that are non-reactive and useful as probes.

Preferred embodiments include dendronic compounds, and methods of their use, where such compounds contain one focal-point group, which may be inert, reactive, ionic or a conjugated substance, and two or more dye components. These compounds may be useful in both free and conjugated forms, as probes, labels, indicators, or sensors. This usefulness may reflect in part an enhancement of one or more of the following: quantum yield, Stokes' shift, extinction coefficients, aqueous solubility, photostability and chemical stability.

One aspect of the current invention overcomes prior art shortcomings by using dendron-based labels that exhibit reduced quenching at higher dye-dendron ratios and which may be covalently labeled to biomolecules and other carriers. Labeling these dendron-reporters to biomolecules does not lead to additional quenching of the dyes on the dendron due to the fact that only one dendron label is in general required to obtain the same sensitivity as one would achieve with multiple dyes directly labeled to the carrier molecule (including proteins, drugs, and other biomolecules).

In addition, the dendronic reporters of this invention offer the possibility to pack the highest possible number of dyes within the smallest volume element possible, enabling FRET donors and acceptors that are capable of expanding the measurable range of FRET to beyond the current limit of around 80 Å (Angstrom). Further, there are other possibilities with these dendron based reporters, e.g., to combine both donors and acceptor molecules on these dendron backbones and to use these FRET based reporters for sensing analytes.

### BRIEF DESCRIPTION OF THE DRAWINGS

Preferred and alternative embodiments are described in detail below with reference to the following drawings:

FIG. 1 is a plot showing the absorption and emission spectrum of Dendron Reporter 1 (DR1).

FIG. 2 is a plot showing the absorption and emission spectrum of Dendron Reporter 4 (DR4).

FIG. 3 is a drawing of exemplary dendron structures.

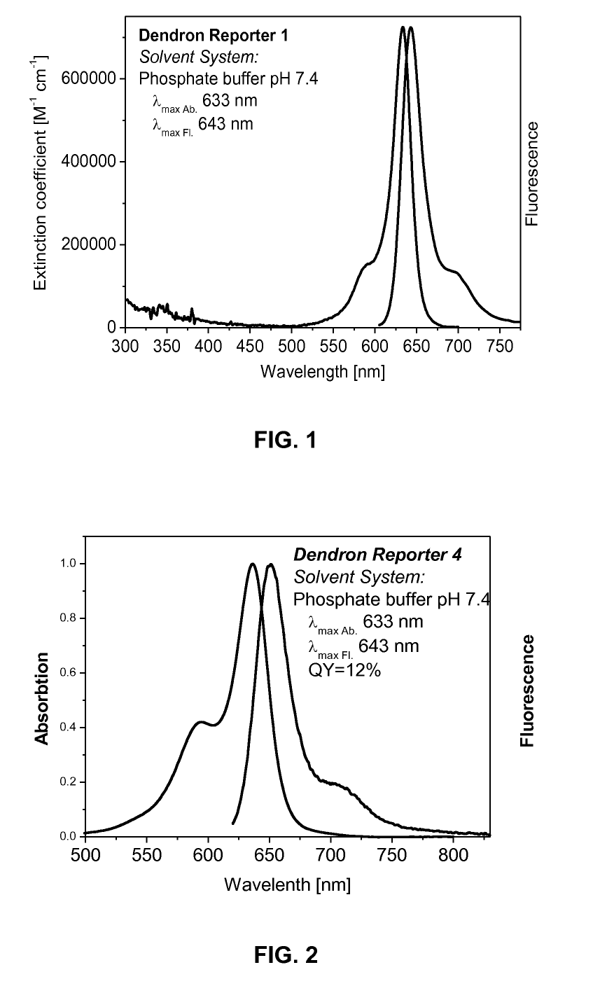

### DETAILED DESCRIPTION OF THE INVENTION

In one aspect of the invention, a dendron reporter may consist of covalently linked dendron units, DU, of the formula

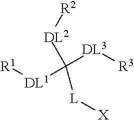

wherein L, DL¹, DL² and DL³ are each independently a single covalent bond or a bivalent linkage that is linear, cyclic or heterocyclic, saturated or unsaturated, having 1-20 non-hydrogen atoms from the group of C, N, P, O and S, in such a way that the linkage contains any combination of ether, thioether, amine, ester, amide bonds; single, double, triple or aromatic carbon-carbon bonds; or carbon-sulfur bonds, carbon-nitrogen bonds, phosphorus-sulfur, nitrogen-nitrogen, nitrogen-oxygen, or aromatic or heteroaromatic bonds;

wherein periphery R¹, R² and R³ substituents are each independently H, alkyl, aryl, alkyl-aryl, amino, amido, ether, hydroxyl, thiol, substituted thiol, carboxyl, carboxylic ester, substituted amino, sulfo, phosphate, phosphonate groups, or a linked dye, provided that at least two of the periphery R¹, R², R³ substituents are linked dyes;

wherein non-periphery R¹, R², R³ substituents are X of another dendron unit DU; and

wherein the focal-point substituent X is a reactive group, ionic group or conjugated substance.

Such a dendron reporter is preferably of generation two or greater (so that ample periphery sites are available for dyes) and preferably of generation less than five (so that the dendron backbone does not add a high mass to the total mass of the dendron reporter). More than half of the periphery R¹, R², R³ groups may be dyes, enabling a high density of dyes per dendron reporter. Where practical, all or substantially all of the periphery R¹, R², R³ groups may be dyes.

An object of the invention is to create reporters with a high density of dyes. The density of dyes conjugated to the dendron may be expressed as the mass to dye ratio, the mass of the dendron reporter including conjugated dyes divided by the number of dyes. Such mass to dye ratio is effective if 5,000 g M⁻¹ or less, more effective at 2,500 g M⁻¹ or less and even more effective at less than 1,000 g M⁻¹. The dendron reporters of the invention enable low mass to dye ratios because the dendron units in the dendron form a structured, low-molecular weight scaffolding to hold a large number of dyes.

Alternatively, the density of dyes may be expressed as the dye to volume ratio, the number of dyes divided by the molecular volume of the entire dendron reporter, the extinction coefficient, or the brightness of the entire dendron reporter. High extinction coefficients such as greater than 20,000 M⁻¹ cm⁻¹, or greater than 200,000 M⁻¹ cm⁻¹, or even greater than 1,000,000 M⁻¹ cm⁻¹ are possible because of the large number of periphery sites available for dyes. Further, high brightness such as greater than 5,000 M⁻¹ cm¹, or greater than 30,000 M⁻¹ cm⁻¹ or even greater than 100,000 M⁻¹ cm⁻¹ are possible because the dyes, when conjugated to the dendron periphery, do not exhibit significant quenching as would occur on a protein.

The conjugation of dye groups may be facilitated by resort to click chemistry, typically a microwave assisted or a simple thermal reaction between a carbon triple bond and an azide in presence of Cu(I). The resulting linkage would be a triazole group (thus each periphery DL¹, DL², and DL³ that links a dye may also include a triazole group).

In another aspect, the invention relates to compositions that include dendronic reporter compounds that are based on the following generic structural elements:

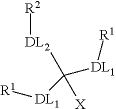

wherein DL₁ and DL₂, is a single covalent bond or a covalent linkage that is linear or branched, cyclic or heterocyclic, saturated or unsaturated, having 1-20 non-hydrogen atoms from the group of C, N, P, O and S, in such a way that the linkage contains any combination of ether, thioether, amine, ester, amide bonds; single, double, triple or aromatic carbon-carbon bonds; or carbon-sulfur bonds, carbon-nitrogen bonds, phosphorus-sulfur, nitrogen-nitrogen, nitrogen-oxygen, or aromatic or heteroaromatic bonds;

wherein R¹ is either a linked dye, L-Dye^{x}, or another dendron unit DU, where DU has the structure:

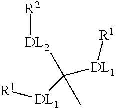

R² is H, alkyl, aryl, alkyl-aryl, or DL₁-R₁, in which case DL₂ is identical to DL₁;

X is selected from L-R^{±}, L-R^{x}, L-S^{c};

provided that a least two of the R residues contain a dye component in such a way that the photophysical properties of the dendron-based reporter are optimized in regards to extinction coefficients and photophysical properties.

In another embodiment, reporters may have the structure:

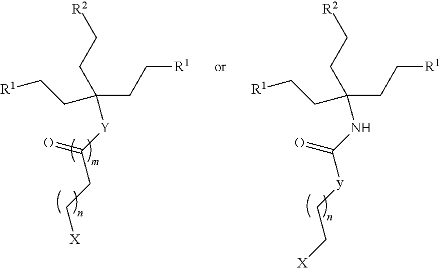

wherein R¹ is either a first linked dye L-Dye^{r}, or COOH, COOEt, or another dendron unit DU, where DU has the structure:

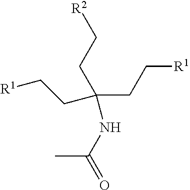

R² is selected from the group consisting of a dendron unit DU, the first linked dye L-Dye^{x}, a second linked dye L-Dye^{x}, or COOH, a reactive group L-R^{x}, an ionic group L-R^{±} and a linked carrier or conjugated substance L-S^{c};

Y is selected from NH, O, S;

n=1-9 and

L is a single covalent bond or is a covalent linkage that is linear or branched, cyclic or heterocyclic, saturated or unsaturated, having 1-20 non-hydrogen atoms from the group of C, N, P, O and S, in such a way that the linkage contains any combination of ether, thioether, amine, ester, amide bonds; single, double, triple or aromatic carbon-carbon bonds; or carbon-sulfur bonds, carbon-nitrogen bonds, phosphorus-sulfur, nitrogen-nitrogen, nitrogen-oxygen or nitrogen-platinum bonds, or aromatic or heteroaromatic bonds;

Dye^{x} and Dye^{y} are selected from a broad range of fluorescent dyes;

X is selected from L-R^{±}, L-R^{x}, L-S^{c};

provided that a least R¹ contains a dye component in such a way that the photophysical properties of the dendron-based reporter are optimized in regards to extinction coefficients and brightness.

In another embodiment, dendron reporters may have the structure:

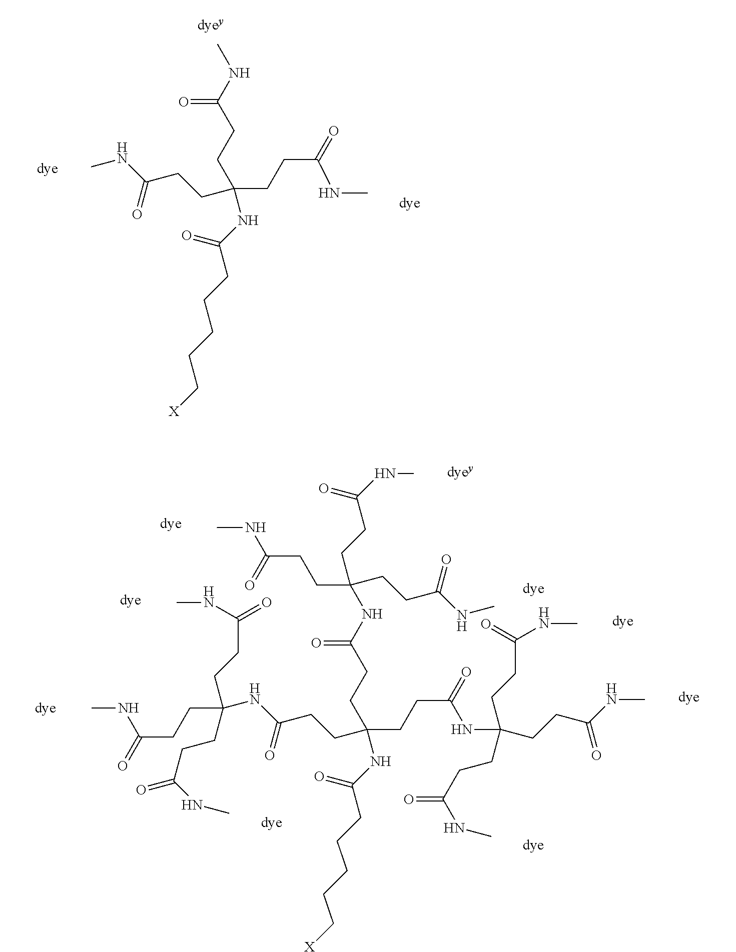

wherein X=CO—NHS, SH, carboxyl, maleimide, iodoacetamide, phosphoramidite, isothiocyanate, alkyl, an ionic group or a linked carrier.

In another embodiment, dendron reporters may have the structure:

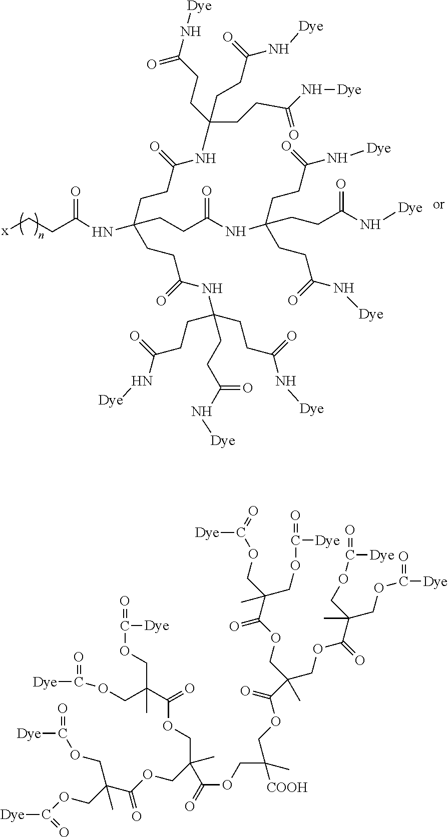

X=S^{c}, R^{±} and R^{x}; alkyl, n is 1-9

R^{x} is NHS, maleimide, iodoacetamide, isothiocyanate, phosphoramidite, carboxyl, amino, sulfonylchloride, azide, alkyne, and DBCO among others;

Dye components may be selected from cyanines, squaraines, oxazines, polyaromatics, heterocyclic dyes, polyaromatic dyes, naphthalic acid derivatives, perylenetetracarboxylic acid derivatives, oxazole derivatives, oxadiazole derivatives, heterocyclic dyes, xanthenes, coumarins, phthalocyanines, porphyrines, BODIPY dyes, rhodamines, metal-ligand complexes (Ru-, Os- and Re-), lanthanide complexes (Eu- and Tb-complexes), styryl dyes, azo dyes, Black Hole Quencher Dyes™, Atto™ dyes, Alexa™ dyes, Seta™ dyes, SeTau™ dyes, Oyster™ dyes, DY dyes, Cy™ dyes, HiLight™ dyes, DyLight™ dyes and IRDyes™ among others.

The main idea behind the synthesis of these novel compositions is to combine as many dye molecules as possible in a small volume element to maximize the sensitivity of these reporters for use in biological assays, sensors and in particular for FRET based applications, where spatial considerations are of importance. This is not the case for the dendrimer reporter molecules described by Weck et al. in Organic Lett. 13 (5), 976-979 (2011), where the dye molecules are distributed over a large volume element, or for the dendrimer reporter molecules described by Albertazzi et al. Mol. Pharmaceutics 73, 680-688 (2010), where on average only about one dye is attached to the dendrimer surface. Such reporters might be useful as sensors but will not be useful as reporters for FRET or polarization based applications, where the size and molecular mass of the reporter play an important role. Further, it is valuable for this type of dendron reporter to include highly charged dye molecules (containing a plurality of ionic groups) that help to reduce the aggregation tendencies of the dyes on the dendron backbone as shown in Examples 1, 3 and 4.

Example 1 and Example 4 demonstrate that high-density dye labeling of the dendron periphery of Behera-type dendrons (4 of 9 possible sites labeled in Example 1, all 9 sites labeled in Example 4) result in no dye quenching effect. Contrary to expectations, labeling of the Behera-type dendron with 4 dyes led to a quantum yield increase from 6% for the free dye to 9% of the labeled dendron. Ordinarily, dye-labeling of proteins can increase quantum yield, but such increase is typically due to the hydrophobic environment of the protein surface. No corresponding increase upon dye-labeling of dendrons is expected because the dendrons are typically too small to provide a sufficient hydrophobic environment. Instead, one would expect to see a decrease in quantum yield due to the close proximity of the dyes on the dendron surface. Based on the molecular mass of the dendron-reporter in Example 1 (MM=4474.2), the extinction coefficient was calculated to be 725,000 M⁻¹ cm⁻¹, which makes this dendron-reporter an extremely bright fluorescent marker, surpassed only by a few other organic reporters, none of which have such a small molecular weight. Table I shows the effect of protein labeling (IgG is immunoglobulin G) on quantum yield and molecular brightness.

TABLE I
Effect of protein labeling on quantum yield and
molecular brightness

| | Extinction | Quantum | Brightness |
|---|---|---|---|
| | Coefficient (ε) | Yield (QY) | (QY × ε) |
| Sample | [M⁻¹ cm⁻¹] | [%] | [M⁻¹ cm⁻¹] |
| DR1 | 725,000 | 9 | 65,250 |
| DR1-IgG (D/P = 1) | 725,000 | 20 | 145,000 |
| Dye-NHS | 280,000 | 6 | 16,800 |
| Dye-IgG (D/P = 1) | 280,000 | 18 | 50,400 |

The brightness of dendron reporters can be further increased several-fold by using more dyes on the dendron periphery (e.g., DR3 has all 9 periphery sites occupied by a dye), or by using dyes with higher extinction coefficients and/or quantum yields as e.g. described in US2010266507A1 and other references provided in this application.

Because of the high brightness and small size, the present invention allows for single-reporter labeling of antibodies. Low label densities cause less interference with antibody function, including affinity for antigens and immune response. Hence the dendron reporters may be used to produce (singly) labeled antibodies superior to conventionally labeled antibodies, even if the same total number of dye units are employed.

Further the mono-reactive dendronic reporters of this invention open a way to increasing the sensitivity for single reporter labeled species such as oligonucleotides and peptides, which are currently limited by the use of either a single fluorescent dye label or a more costly and complex signal transduction system (e.g. biotin-labeled streptavidin).

Another aspect of this invention is to generate internal FRET based compositions, wherein at least one dye molecule (e.g. R²) is a luminescent donor which is combined with a luminescent or non-luminescent acceptor (R¹) on the same dendron backbone. It is understood that the positions of the donor and acceptor are exchangeable.

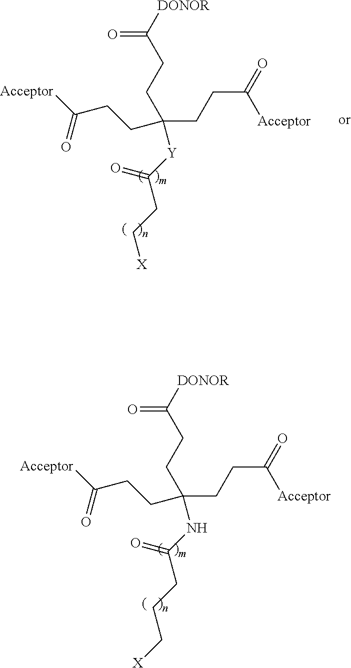

It is understood that the distance between donors and acceptors can be conveniently controlled by changing the generation of the Behera's-type dendron or by changing the length of one of the 3 branches in the dendron backbone as described in Macromolecules 2003, 36, 4345-4354. These reporters are useful as labels as well as probes and either can be covalently attached to a carrier molecule via X or used directly without the need of a carrier (X is non-reactive).

Another aspect of the invention is the use of this concept to generate ratiometric sensors by combination of an analyte-sensitive dye (R¹=Sensor) with a reference dye (R²=Ref) on the same dendron backbone:

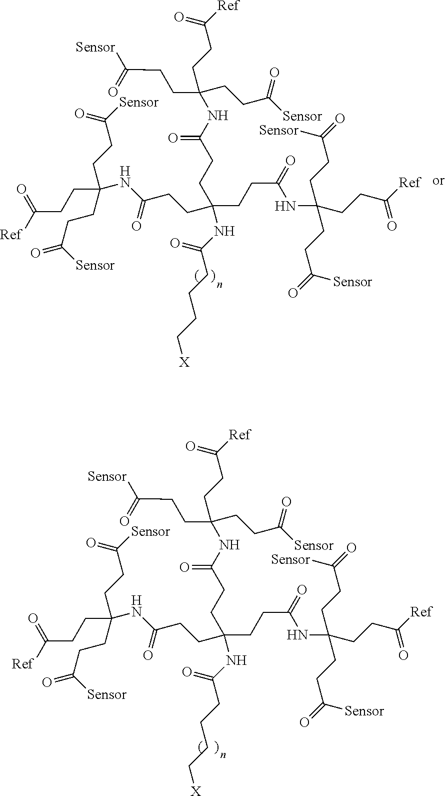

where X=L-R^{±}, L-R^{x}, L-S^{c}; n is 0-10.

These modifications are possible because of the possibility to synthesize unsymmetrically substituted dendrons as e.g. compound 18 in Macromolecules 2003, 36, 4345-4354, which allows introducing 2 or more different reactive end-groups into the dendronic backbone:

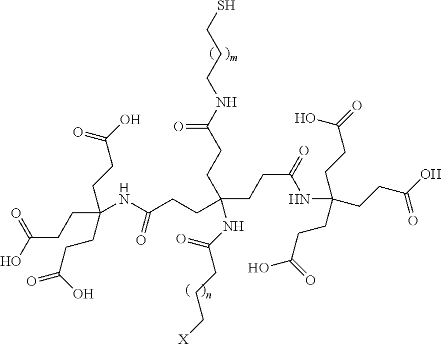

where X=L-R^{±}, L-R^{x}, L-S and

n and m are 0-10.

Overview of Structures

One exemplary dendron reporter has the following structure:

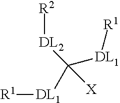

wherein DL₁ and DL₂, is a single covalent bond or a covalent linkage that is linear or branched, cyclic or heterocyclic, saturated or unsaturated, having 1-20 non-hydrogen atoms from the group of C, N, P, O and S, in such a way that the linkage contains any combination of ether, thioether, amine, ester, amide bonds; single, double, triple or aromatic carbon-carbon bonds; or carbon-sulfur bonds, carbon-nitrogen bonds, phosphorus-sulfur, nitrogen-nitrogen, nitrogen-oxygen, or aromatic or heteroaromatic bonds;

wherein periphery R¹ is H, alkyl, aryl, alkyl-aryl, L-Dye^{r} or otherwise another dendron unit DU, where DU has the structure

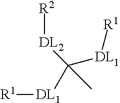

where periphery R² is H, alkyl, aryl, alkyl-aryl, or DL₁-R₁, in which case DL₂ is DL₁;

where X is selected from L-R^{±}, L-R^{x}, L-S^{c};

provided that a least two of the periphery R¹ and R² substituents contain a dye component in such a way that the photophysical properties of the dendron-based reporter are optimized in regards to extinction coefficients and brightness.

Another exemplary dendron reporter has the structure:

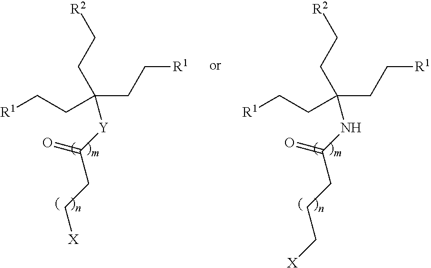

wherein each R¹ is independently a dendron unit, —CONH—C(CH₂CH₂R¹)₃, COOH, COOEt, or L-Dye^{x};

wherein each R² is independently a dendron unit, —CONH—C(CH₂CH₂R²)₃, L-Dye^{x}, L-Dye^{y}, COOH, COOEt, a reactive group, an ionic group or a linked carrier, provided that at least one R¹ includes Dye^{x} and at least one R² includes Dye^{x} or Dye;

wherein Y is selected from NH, O, S;

wherein n=1-9;

wherein X is selected from L-R^{±} (a linked ionic group), L-Rx (a linked reactive group) or L-S^{c} (a linked carrier or conjugated substance); and

wherein L is a single covalent bond or is a covalent linkage that is linear or branched, cyclic or heterocyclic, saturated or unsaturated, having 1-20 non-hydrogen atoms from the group of C, N, P, O and S, in such a way that the linkage contains any combination of ether, thioether, amine, ester, amide bonds; single, double, triple or aromatic carbon-carbon bonds; or carbon-sulfur bonds, carbon-nitrogen bonds, phosphorus-sulfur, nitrogen-nitrogen, nitrogen-oxygen or nitrogen-platinum bonds, or aromatic or heteroaromatic bonds.

Dendron reporters may be built from different components as long as the dendron structure is maintained in the final reporter molecule. For example, dendron reporters may be based upon the backbone shown below (Sigma-Aldrich product 686670):

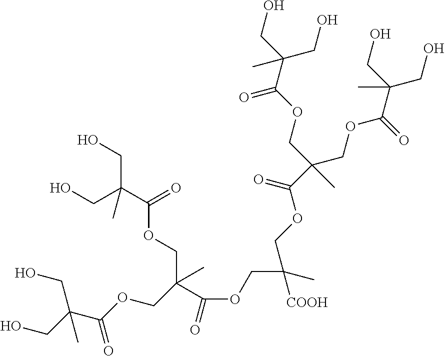 [SMILES: CC(COC(=O)C(C)(COC(=O)C(C)(CO)CO)COC(=O)C(C)(CO)CO)(COC(=O)C(C)(COC(=O)C(C)(CO)CO)COC(=O)C(C)(CO)CO)C(=O)O]

This structure can be modified according to the following scheme and the reactive carboxyl group can be used for linking to various carrier groups as described above.

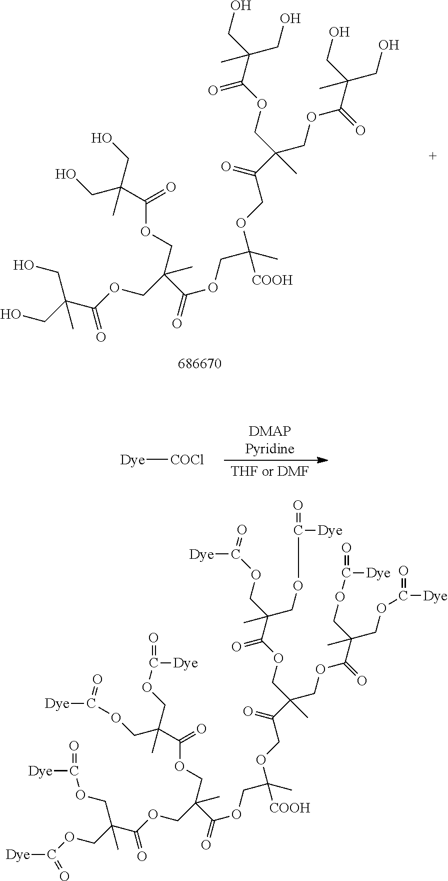

Dye Compounds

The dendronic reporter compounds may be luminescent or non-luminescent and may have utility as non-fluorescent reporters in absorption based assays or as luminescent probes or as labels in photoluminescence assays and methods, as discussed above. Quenchers can be used as reporters in photo-acoustic measurements. The fluorescent or non-fluorescent dye component in these reporters can be chosen very broadly from various classes of dyes:

Cyanines, squaraines, squaraine rotaxanes, ozazines, polyaromatics, heterocyclic dyes, heteroaromatic dyes, xanthene dyes, coumarins, phthalocyanines, porphyrines, BODIPY dyes, rhodamines, metal-ligand complexes (Ru-, Os- and Re-), lanthanide complexes (Eu- and Tb-complexes), and including dyes and labels that are described in Richard P. Haugland, HANDBOOK OF FLUORESCENT PROBES AND RESEARCH CHEMICALS (6^{th} ed. 1996).

The sensors, dyes and reactive versions of these dye classes, including groups for functionalization of these dendrons are described in the following references: Richard P. Haugland, HANDBOOK OF FLUORESCENT PROBES AND RESEARCH CHEMICALS (6^{th} ed. 1996); Greg Hermanson, Bioconjugate Techniques 2^{nd} Ed., Academic Press, Elsevier 2008; Journal of Photochemistry and Photobiology A: Chemistry 190 (2007) 1-8; Tetrahedron Letters 47 (2006) 8279-8284; Anal. Biochem 217, 197-204 (1994); Anal. Biochem 288, 62-75 (2001); Bioconjugate Chem., Vol. 13, No. 3, (2002); Bioconjugate Chem. 20, 1807-1812 (2009); Bioconjugate Chem. 1996, 7, 356-362; Anal. Biochem 247, 216-222 (1997); Bioconjugate Chem. 2000, 11, 533-536; Bioconjugate Chem. 1999, 10, 925-931; Bioconjugate Chem. 4, 105-111 (1993); Anal. Biochem. 227, 140-147 (1995); Anal. Biochem. 232, 24-30 (1995); Spectrochimica Acta Part A 61 (2005) 109-116; Inorg. Chem. 1985, 24, 2755-2763; Anal. Biochem 342 (2005) 111-119; J. AM. CHEM. SOC. 2004, 126, 712-713; J. AM. CHEM. SOC. 2007, 129, 77-83; J. Org. Chem. 2009, 74, 3183-3185; Tetrahedron Letters 44 (2003) 3975-3978; Bioconjugate Chem. 2003, 14, 1048-1051; Cytometry 29:328-339 (1997); Angew. Chem. 2007, 119, 5624-5627; and further in the following patents and patent applications: US2005214810A1; US5569587A1 U.S. Pat. No. 6,977,305; W00075237A3; U.S. Pat. No. 5,569,766; U.S. Pat. No. 5,714,386; W00220670A1; W02004055117A2; US2010197030A1; US2006292658A1; WO06047452; U.S. Pat. No. 6,617,458B2; US2002147354; US2006177857A1; U.S. Pat. No. 7,745,640B2; US20060229441; U.S. Pat. No. 6,995,262B1; U.S. Pat. No. 6,417,402B1; US2006/0166368A1; U.S. Pat. No. 7,566,783B2; W08703589A1; W02007098182A2; W003082988A1; US2007021621A1; EP1319047B1; U.S. Pat. No. 6,995,274B2; WO2010054183; US2005214810A1; U.S. Pat. No. 5,569,587A1 including those references cited in other sections of this document.

Groups and Substituents

The compounds of this invention may include a variety of different groups or substituents:

A hydrophilic group is any group which increases solubility of a compound in aqueous media. These groups include, but are not limited to, sulfo, sulfonic, phosphate, phosphonate, phosphonic, carboxylate, boronic, ammonium, cyclic ammonium, hydroxy, alkoxy, ester, polyethylene glycol, polyester, glycoside, and saccharide groups.

A hydrophobic group is a group (e.g. aliphatic groups) that decreases the solubility of a compound in aqueous media.

Reactive Groups R^{x}

The dendron reporters of this invention may include one or more reactive groups, where a reactive group generally is a group capable of forming a covalent attachment with another molecule or substrate. Such other molecules or substrates may include proteins, carbohydrates, nucleic acids, and plastics, among others. Reactive groups vary in their specificity, and may preferentially react with particular functionalities and molecule types. Thus, reactive compounds generally include reactive groups chosen preferentially to react with functionalities found on the molecule or substrate with which the reactive compound is intended to react.

The compounds of the invention are optionally substituted, either directly or via a substituent, by one or more chemically reactive functional groups that may be useful for covalently attaching the compound to a desired substance. Each reactive group, or R^{x}, may be bound to the compound directly by a single covalent bond, or may be attached via a covalent spacer or linkage, L, and may be depicted as L-R^{x}.

The reactive functional group of the invention Rx may be selected from the following functionalities, among others: activated carboxylic esters, azides, acyl halides, acyl nitriles, acyl nitriles, aldehydes, ketones, alkyl halides, alkyl sulfonates, anhydrides, aryl halides, aziridines, boronates, carboxylic acids, carbodiimides, diazoalkanes, epoxides, haloacetamides, halotriazines, imido esters, isocyanates, isothiocyanates, maleimides, phosphoramidites, silyl halides, sulfonate esters, and sulfonyl halides.

In particular, the following reactive functional groups, among others, are particularly useful for the preparation of labeled molecules or substances, and are therefore suitable reactive functional groups for the purposes of the reporter compounds:

a) N-hydroxysuccinimide (NHS) esters, isothiocyanates, and sulfonylchlorides, which form stable covalent bonds with amines, including amines in proteins and amine-modified nucleic acids;

b) Iodoacetamides and maleimides, which form covalent bonds with thiol-functions, as in proteins;

c) Carboxyl functions and various derivatives, including N-hydroxybenztriazole esters, thioesters, p-nitrophenyl esters, alkyl, alkenyl, alkynyl, and aromatic esters, and acyl imidazoles;

d) Alkylhalides, including iodoacetamides and chloroacetamides;

e) Hydroxyl groups, which can be converted into esters, ethers, and aldehydes;

f) Aldehydes and ketones and various derivatives, including hydrazones, oximes, and semicarbozones;

g) Isocyanates, which may react with amines;

h) Activated C═C double-bond-containing groups, which may react in a Diels-Alder reaction to form stable ring systems under mild conditions;

i) Thiol groups, which may form disulfide bonds and react with alkylhalides (such as iodoacetamide);

j) Alkenes, which can undergo a Michael addition with thiols, e.g., maleimide reactions with thiols;

k) Phosphoramidites, which can be used for direct labeling of nucleosides, nucleotides, and oligonucleotides, including primers on solid or semi-solid supports;

l) Primary amines that may be coupled to variety of groups including carboxyl, aldehydes, ketones, and acid chlorides, among others;

m) Boronic acid derivatives which may react with sugars;

n) Pyrylium moieties which react with primary amines;

o) Haloplatinates which form stable platinum complexes with amines, thiols and heterocycles;

p) Aryl halides which may react with thiols and amines;

q) Azides which spontaneously react with triple bonds to triazoles in presence of Cu(I);

r) Dibenzylazacyclo-octen (DBCO) and other cyclo-alkyn groups which react with azides in a copper free strain-mediated cycloaddition reaction to triazoles.

R Groups

The R moieties associated with the dendronic reporter may include any of a number of groups including but not limited to alicyclic groups, aliphatic groups, aromatic groups, and heterocyclic rings, as well as substituted versions thereof.

Aliphatic groups include groups of organic compounds characterized by straight- or branched-chain arrangement of the constituent carbon atoms. Aliphatic hydrocarbons comprise three subgroups: (1) paraffins (alkanes), which are saturated and comparatively unreactive; (2) olefins (alkenes or alkadienes), which are unsaturated and quite reactive; and (3) acetylenes (alkanes), which contain a triple bond and are highly reactive. In complex structures, the chains may be branched or cross-linked and may contain one or more heteroatoms (such as polyethers and polyamines, among others).

Alicyclic groups include hydrocarbon substituents that incorporate closed rings. Alicyclic substituents may include rings in boat conformations, chair conformations, or resemble bird cages. Most alicyclic groups are derived from petroleum or coal tar, and many can be synthesized by various methods. Alicyclic groups may optionally include heteroalicyclic groups that include one or more heteroatoms, typically nitrogen, oxygen, or sulfur. These compounds have properties resembling those of aliphatics and should not be confused with aromatic compounds having the hexagonal benzene ring. Alicyclics may comprise three subgroups: (1) cycloparaffins (saturated), (2) cycloolefins (unsaturated with two or more double bonds), and (3) cycloacetylenes (cyclynes) with a triple bond. The best-known cycloparaffins (sometimes called naphthenes) are cyclopropane, cyclohexane, and cyclopentane; typical of the cycloolefins are cyclopentadiene and cyclooctatetraene.

Aromatic groups may include groups of unsaturated cyclic hydrocarbons containing one or more rings. A typical aromatic group is benzene, which has a 6-carbon ring formally containing three double bonds in a delocalized ring system. Aromatic groups may be highly reactive and chemically versatile. Most aromatics are derived from petroleum and coal tar. Heterocyclic rings include closed-ring structures, usually of either 5 or 6 members, in which one or more of the atoms in the ring is an element other than carbon, e.g., sulfur, nitrogen, etc. Examples include pyridine, pyrole, furan, thiophene, and purine. Some 5-membered heterocyclic compounds exhibit aromaticity, such as furans and thiophenes, among others, and are analogous to aromatic compounds in reactivity and properties.

Any substituent of the compounds of the invention, including any aliphatic, alicyclic, or aromatic group, may be further substituted one or more times by any of a variety of substituents, including without limitation, F, Cl, Br, I, carboxylic acid, sulfonic acid, CN, nitro, hydroxy, phosphate, phosphonate, sulfate, cyano, azido, amine, alkyl, alkoxy, trialkylammonium or aryl. Aliphatic residues can incorporate up to six heteroatoms selected from N, O, S. Alkyl substituents include hydrocarbon chains having 1-22 carbons, more typically having 1-6 carbons, sometimes called “lower alkyl”.

As described in WO01/11370, sulfonamide groups such as —(CH₂)ₙ—SO₂—NH—SO₂—R, —(CH₂)ₙ—CONH—SO₂—R, —(CH₂)ₙ—SO₂—NH—CO—R, and —(CH₂)ₙ—SO₂NH—SO₃H, where R is aryl or alkyl and n=1-6, can be used to reduce the aggregation tendency and have positive effects on the photophysical properties of cyanines and related dyes. Such groups might also be useful to reduce the aggregation tendencies of dyes labeled to the dendron backbone of this invention.

Where a substituent R is further substituted by a functional group that is formally electronically charged, such as for example a carboxylic acid, sulfonic acid, phosphoric acid, phosphonate or a quaternary ammonium group, the resulting ionic substituent may serve to increase the overall hydrophilicity of the compound. Examples of electronically charged functional groups include —PO₃²⁻, —O—PO₃², —PO₃R^{m-}, —O—PO₃R^{m-}, —C₆H₄—SO₃⁻, —C₆H₄—PO₃⁻, pyridylium, pyrylium, —SO₃⁻, —O—SO₃⁻, —COO⁻ and ammonium, among others.

As used herein, functional groups such as “carboxylic acid,” “sulfonic acid,” and “phosphoric acid” include the free acid moiety as well as the corresponding metal salts of the acid moiety, and any of a variety of esters or amides of the acid moiety, including without limitation alkyl esters, aryl esters, and esters that are cleavable by intracellular esterase enzymes, such as alpha-acyloxyalkyl ester (for example acetoxymethylene esters, among others). Further these esters might contain additional reactive or ionic groups and linked carriers.

The compounds of the invention may be depicted in structural descriptions as possessing an overall charge, it is to be understood that the compounds depicted include an appropriate counter ion or counter ions to balance the formal charge present on the compound. Further, the exchange of counter ions is well known in the art and readily accomplished by a variety of methods, including ion-exchange chromatography and selective precipitation, among others.

Carriers and Conjugated Substances S^{c}

The reporter compounds of the invention, including synthetic precursor compounds, may be covalently or non-covalently associated with one or more carriers or substances. Covalent association may occur through various mechanisms, including a reactive functional group as described above, and may involve a covalent linkage, L, separating the compound or precursor from the associated carrier or substance (which may therefore be referred to as L-S′).

The covalent linkage L binds the reactive group R^{x}, the conjugated substance S^{c} or the ionic group R^{±} to the dye molecule, either directly (L is a single bond) or with a combination of stable chemical bonds, that include single, double, triple or aromatic carbon-carbon bonds; carbon-sulfur bonds, carbon-nitrogen bonds, phosphorus-sulfur bonds, nitrogen-nitrogen bonds, nitrogen-oxygen or nitrogen-platinum bonds, or aromatic or heteroaromatic bonds; L includes ether, thioether, carboxamide, sulfonamide, urea, urethane or hydrazine moieties. Preferable L include a combination of single carbon-carbon bonds and carboxamide or thioether bonds.

Where the substance is associated non-covalently, the association may occur through various mechanisms, including incorporation of the compound or precursor into or onto a solid or semisolid matrix, such as a bead or a surface, or by nonspecific interactions, such as hydrogen bonding, ionic bonding, or hydrophobic interactions (such as Van der Waals forces). The associated carrier may be selected from the group consisting of polypeptides, polynucleotides, polysaccharides, beads, microplate well surfaces, metal surfaces, semiconductor and non-conducting surfaces, nano-particles, and other solid surfaces.

The associated or conjugated substance may be associated with or conjugated to more than one reporter compound, which may be the same or different. Generally, methods for the preparation of dye-conjugates of biological substances are well-known in the art. See, for example, Haugland et al., MOLECULAR PROBES HANDBOOK OF FLUORESCENT PROBES AND RESEARCH CHEMICALS, Eighth Edition (1996), which is hereby incorporated by reference. Typically, the association or conjugation of a chromophore or luminophore to a substance imparts the spectral properties of the chromophore or luminophore to that substance.

Useful substances for preparing conjugates according to the present invention include, but are not limited to, amino acids, peptides, proteins, nucleosides, nucleotides, nucleic acids, carbohydrates, lipids, ion-chelators, non-biological polymers, cells, and cellular components. The substance to be conjugated may be protected on one or more functional groups in order to facilitate the conjugation, or to insure subsequent reactivity.

Where the substance is a peptide, the peptide may be a dipeptide or larger, and typically includes 5 to 36 amino acids. Where the conjugated substance is a protein, it may be an enzyme, an antibody, lectin, protein A, protein G, hormones, or a phycobiliprotein. The conjugated substance may be a nucleic acid polymer, such as for example DNA oligonucleotides, RNA oligonucleotides (or hybrids thereof), or single-stranded, double-stranded, triple-stranded, or quadruple-stranded DNA, or single-stranded or double-stranded RNA.

Another class of carriers includes carbohydrates that are polysaccharides, such as dextran, heparin, glycogen, starch and cellulose.

Where the substance is an ion chelator, the resulting conjugate may be useful as an ion indicator (calcium, sodium, magnesium, zinc, potassium and other important metal ions) particularly where the optical properties of the reporter-conjugate are altered by binding a target ion. Preferred ion-complexing moieties are crown ethers (U.S. Pat. No. 5,405,957) and BAPTA chelators (U.S. Pat. No. 5,453,517).

The associated or conjugated substance may be a member of a specific binding pair, and therefore useful as a probe for the complementary member of that specific binding pair, each specific binding pair member having an area on the surface or in a cavity which specifically binds to and is complementary with a particular spatial and polar organization of the other. The conjugate of a specific binding pair member may be useful for detecting and optionally quantifying the presence of the complementary specific binding pair member in a sample, by methods that are well known in the art.

Representative specific binding pairs may include ligands and receptors, and may include but are not limited to the following pairs: antigen-antibody, biotin-avidin, biotin-streptavidin, IgG-protein A, IgG-protein G, carbohydrate-lectin, enzyme-enzyme substrate; ion-ion-chelator, hormone-hormone receptor, protein-protein receptor, drug-drug receptor, DNA-antisense DNA, and RNA-antisense RNA.

Preferably, the associated or conjugated substance includes antibodies, proteins, carbohydrates, nucleic acids, and non-biological polymers such as plastics, metallic nanoparticles such as gold, silver and carbon nanostructures among others. Further carrier systems include cellular systems (animal cells, plant cells, bacteria). Reactive dyes can be used to label groups at the cell surface, in cell membranes, organelles, or the cytoplasm.

Finally these compounds can be linked to small molecules such as amino acids, vitamins, drugs, haptens, toxins, environmental pollutants. Another important ligand is tyramine, where the conjugate is useful as a substrate for horseradish peroxidase. Additional embodiments are described in U.S. Patent Application Publication No. US 2002/0077487.

Synthesis

The synthesis of dendronic precursors is described in Organic Lett. 9 (11), 2051-2054 (2007) or in J. Org. Chem. 56, 7162-7167 (1991) and is further described below. There are several references that also describe dendrons containing different reactive groups that are suitable starting materials for dendron based labels as described in Macromolecules 2003, 36, 4345-4354 and J. Org. Chem. 1991, 56, 7162-7167.

The fluorescent or non-fluorescent dye component in these reporters can be chosen very broadly from various classes of dyes:

Cyanines, squaraines, squaraine rotaxanes, ozazines, polyaromatics, xanthenes, coumarins, phthalocyanines, porphyrines, BODIPY dyes, polyaromatic dyes, naphthalic acid dyes, perylenetetracarboxylic acid dyes, oxazole dyes, oxadiazole dyes, a heterocyclic dye, a rhodamine dye, metal-ligand complexes (Ru-, Os- and Re-), lanthanide complexes (Eu- and Tb-complexes) among other classes of dyes.

These sensors, dyes and reactive versions of these dye classes, including groups for functionalization of these dendrons are described in the following references: Richard P. Haugland, HANDBOOK OF FLUORESCENT PROBES AND RESEARCH CHEMICALS (6^{th} ed. 1996); Greg Hermanson, Bioconjugate Techniques 2^{nd} Ed., Academic Press, Elsevier 2008; Journal of Photochemistry and Photobiology A: Chemistry 190 (2007) 1-8; Tetrahedron Letters 47 (2006) 8279-8284; Anal. Biochem 217, 197-204 (1994); Anal. Biochem 288, 62-75 (2001); Bioconjugate Chem., Vol. 13, No. 3, (2002); Bioconjugate Chem. 20, 1807-1812 (2009); Bioconjugate Chem. 1996, 7, 356-362; Anal. Biochem 247, 216-222 (1997); Bioconjugate Chem. 2000, 11, 533-536; Bioconjugate Chem. 1999, 10, 925-931; Bioconjugate Chem. 4, 105-111 (1993); Anal. Biochem. 227, 140-147 (1995); Anal. Biochem. 232, 24-30 (1995); Spectrochimica Acta Part A 61 (2005) 109-116; Inorg. Chem. 1985, 24, 2755-2763; Anal. Biochem 342 (2005) 111-119; J. AM. CHEM. SOC. 2004, 126, 712-713; J. AM. CHEM. SOC. 2007, 129, 77-83; J. Org. Chem. 2009, 74, 3183-3185; Tetrahedron Letters 44 (2003) 3975-3978; Bioconjugate Chem. 2003, 14, 1048-1051; Cytometry 29:328-339 (1997); Angew. Chem. 2007, 119, 5624-5627 and further in the following patents and patent applications: U.S. Pat. No. 6,977,305; W00075237A3; U.S. Pat. No. 5,569,766; U.S. Pat. No. 5,714,386; W00220670A1; W02004055117A2; US2010197030A1; US20040260072A1; US2006292658A1; WO06047452; U.S. Pat. No. 6,617,458B2; US2002147354; US2006177857A1; U.S. Pat. No. 7,745,640B2; US20060229441; U.S. Pat. No. 6,995,262B1; U.S. Pat. No. 6,417,402B1; US20060166368A1; U.S. Pat. No. 7,566,783B2; W08703589A1; W02007098182A2; W003082988A1; U.S. Pat. No. 5,438,135; U.S. Pat. No. 6,402,037; US2007021621A1; US2006223076; U.S. Pat. No. 4,945,171; 20060199242A1; US 20080048111A1; 20070077549A1; EP1319047B1; U.S. Pat. No. 6,995,274 B2; WO2010054183; US2005214810A1; U.S. Pat. No. 5,569,587A1 including those references cited in other parts of this document.

The covalent linkage between the dye component and the dendron component can be very broadly chosen from different linking moieties as described above. Preferred linking components are NHS esters and amines, maleimides and thiol-groups and in particular click chemistry type reactions of azides with triple bonds forming stable triazole-bonds. A large number of fluorescent dyes are commercially available with these functional groups.

### Example 1

### Synthesis of Precursors and Intermediates

The synthesis of dendron precursors for the synthesis of these reporters are described in Organic Lett. 9 (11), 2051-2054 (2007) or in JOC 56, 7162-7167 (1991). The synthesis of unsymmetrical substituted dendron structures are described in Macromolecules 2003, 36, 4345-4354 and J. Org. Chem. 1991, 56, 7162-7167. The starting material, D1 dendron tert-butyl ester, was purchased from Frontier Scientific catalog #NTN12046. Starting materials for dendrons with an —N═C═O function for conversion to NH—CO—NH, NH—CO—O— and NH—CO—S— groups are commercially available from Frontier Scientific catalog #NTN 1962 (other starting materials are available from Sigma-Aldrich as described above).

### Example 2

### Synthesis of Dendron Reporter 1 (DR1)

Hydrolysis of Dendron tert-butyl ester

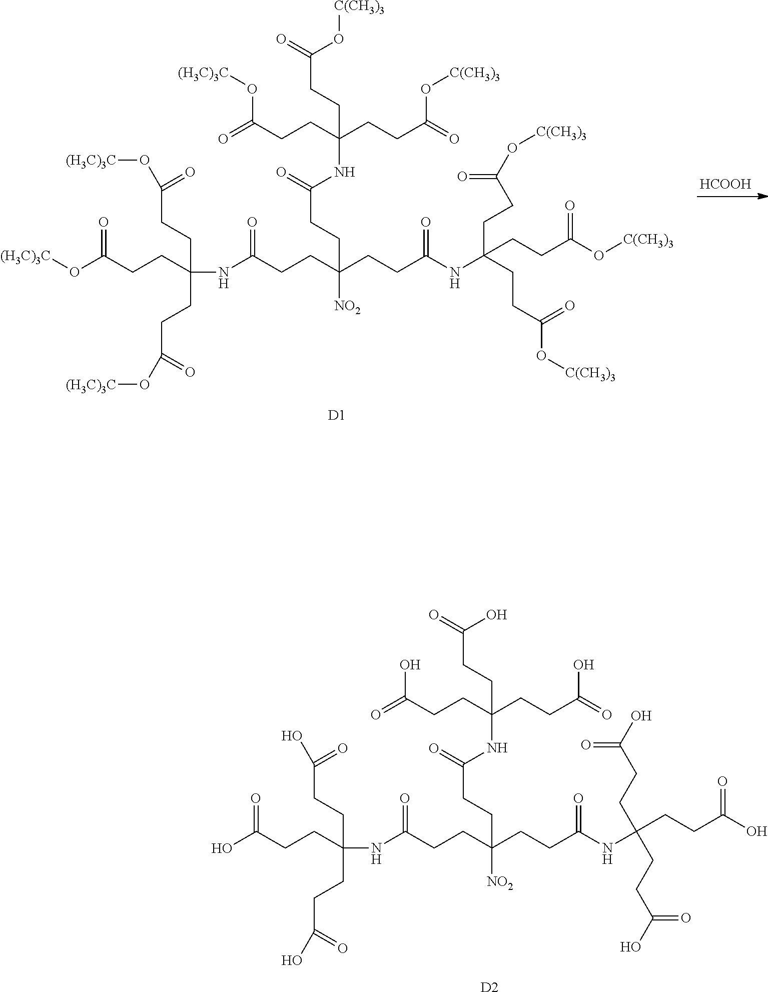 [SMILES: CC(C)(C)OC(=O)CCC(CCC(=O)OC(C)(C)C)(CCC(=O)OC(C)(C)C)NC(=O)CCC(CCC(=O)NC(CCC(=O)OC(C)(C)C)(CCC(=O)OC(C)(C)C)CCC(=O)OC(C)(C)C)(CCC(=O)NC(CCC(=O)OC(C)(C)C)(CCC(=O)OC(C)(C)C)CCC(=O)OC(C)(C)C)[N+](=O)[O-] ; "HCOOH" ; "D1" ; O=C(O)CCC(CCC(=O)O)(CCC(=O)O)NC(=O)CCC(CCC(=O)NC(CCC(=O)O)(CCC(=O)O)CCC(=O)O)(CCC(=O)NC(CCC(=O)O)(CCC(=O)O)CCC(=O)O)[N+](=O)[O-] ; "D2"]

A solution of 50 mg (34 μmol) of the dendron tert-butyl ester (D1) and 0.5 mL of 94% formic acid was stirred at room temperature for 25 h. The mixture was concentrated in vacuo, triturated with ether and dried in vacuum to afford the acid of the dendron in quantitative yield (D2).

Synthesis of the Dendron NHS Ester

1.6 mg (1.66 μmol) of hydrolyzed dendron (D2) and 36 mg (120 μmol) of 0-(N-succinimidyl)-N,N,N′,N′-tetramethyluronium tetrafluoroborate (TSTU) were dissolved in 750 μL of dimethylformamide (DMF), then 29 μL (207 μmol) of N,N-diisopropyl ethyl amine (DIPEA) were added. The solution was stirred at room temperature for 1 hour and then used for the reaction with the amino-compound without isolation of NHS ester (D3).

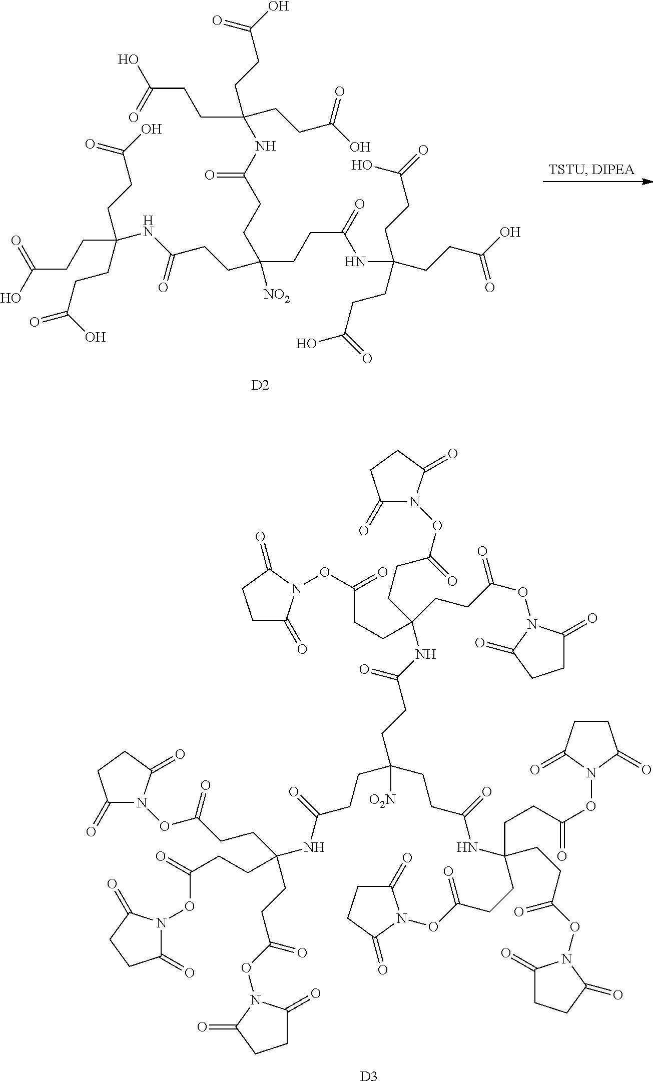 [SMILES: O=C(O)CCC(CCC(=O)O)(CCC(=O)O)NC(=O)CCC(CCC(=O)NC(CCC(=O)O)(CCC(=O)O)CCC(=O)O)(CCC(=O)NC(CCC(=O)O)(CCC(=O)O)CCC(=O)O)[N+](=O)[O-] ; "D2" ; O=C(CCC(CCC(=O)NC(CCC(=O)ON1C(=O)CCC1=O)(CCC(=O)ON1C(=O)CCC1=O)CCC(=O)ON1C(=O)CCC1=O)(CCC(=O)NC(CCC(=O)ON1C(=O)CCC1=O)(CCC(=O)ON1C(=O)CCC1=O)CCC(=O)ON1C(=O)CCC1=O)[N+](=O)[O-])NC(CCC(=O)ON1C(=O)CCC1=O)(CCC(=O)ON1C(=O)CCC1=O)CCC(=O)ON1C(=O)CCC1=O ; "D3"]

Synthesis of Dendron Reporter 1 (DR1)

21.5 mg (25 μmol) of amine-modified squaraine dye (Sq1) were dissolved in 500 μL of DMF. The amine-modification is typically achieved by reacting the NHS-ester of the dye with ethylenediamine as described in Bioconjugate Chem. 20, 1807-1812 (2009). This solution was added to the solution of the dendron NHS ester (D3) in 750 μL, obtained from 1.6 mg (1.66 μmol) of dendron (D2). Reaction mixture was stirred for 15 h at room temperature. Then the product was precipitated with 50 mL of methyl tert-butyl ether (MTBE) and cooled in the freezer. The blue precipitate was filtered off, washed with ether and purified by column chromatography on Lichroprep RP-18 (gradient 0-11% acetonitrile in water) to yield 3.9 mg of N,N-diisopropyl ethyl ammonium salt of Dendron Reporter 1 (DR1), containing four dye molecule on one dendron. ¹H-NMR (200 MHz, DMSO-d₆), δ, ppm: 7.85-8.10 (9H, CONH and NH (DIPEA), m), 7.70-7.83 (3H, CONH, m), 7.67 (4H, arom H, s), 7.65 (4H, arom H, d, 8.4 Hz), 7.63 (4H, arom H, s), 7.61 (4H, arom H, d, 7.8 Hz), 7.31 (4H, arom H, d, 8.5 Hz), 7.26 (4H, arom H, d, 8.4 Hz), 5.86 (4H, CH, s), 5.80 (4H, CH, s), 4.00-4.20 (16H, NCH₂, m), 3.01 (16H, NCH₂CH₂N, broad s), 2.57-2.67 (8H, CH₂SO₃H, m), 2.38 (8H, CH₂CO (dye), m), 2.05-2.29 (24H, CH₂ (dendron), m), 1.70-1.89 (32H, CH₂ (dendron and dye), m), 1.68 (12H, CH₃, s), 1.67 (24H, CH₃, s), 1.43-1.65 (16H, CH₂ (dye), m), 0.93-1.40 (43H, CH₃ (DIPEA) and CH₃ (ethyl), CH₂ (dye), m), 0.27-0.90 (8H, CH₂, m), signals CH (isopropyl) and CH₂ (ethyl) of DIPEA are hidden under signal of water. MALDI-TOF m/z calculated for (C₂₀₄H₂₇₁N₂₁O₆₇S₁₂)⁺: 4474.23. found: 4474.2.

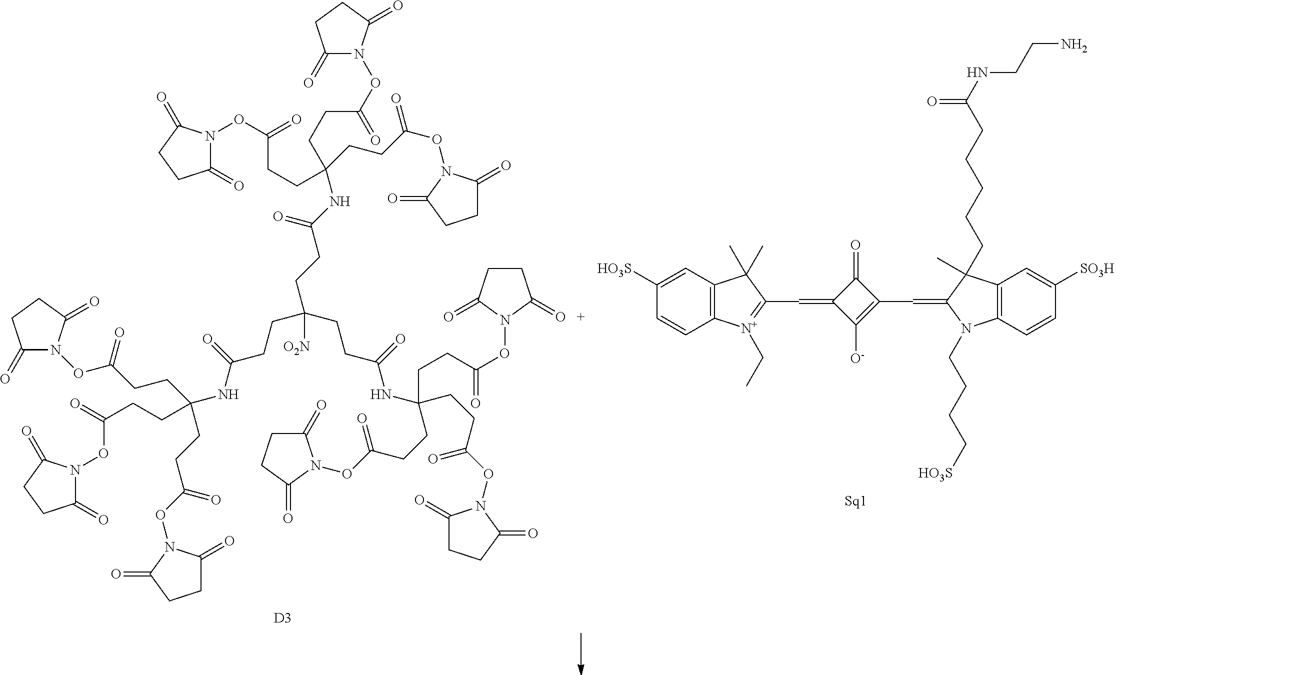 [SMILES: O=C(CCC(CCC(=O)NC(CCC(=O)ON1C(=O)CCC1=O)(CCC(=O)ON1C(=O)CCC1=O)CCC(=O)ON1C(=O)CCC1=O)(CCC(=O)NC(CCC(=O)ON1C(=O)CCC1=O)(CCC(=O)ON1C(=O)CCC1=O)CCC(=O)ON1C(=O)CCC1=O)[N+](=O)[O-])NC(CCC(=O)ON1C(=O)CCC1=O)(CCC(=O)ON1C(=O)CCC1=O)CCC(=O)ON1C(=O)CCC1=O ; CC[N+]1=C(C=C2C(=O)C(C=C3N(CCCCS(=O)(=O)O)c4ccc(S(=O)(=O)O)cc4C3(C)CCCCCC(=O)NCCN)=C2[O-])C(C)(C)c2cc(S(=O)(=O)O)ccc21 ; "D3"]

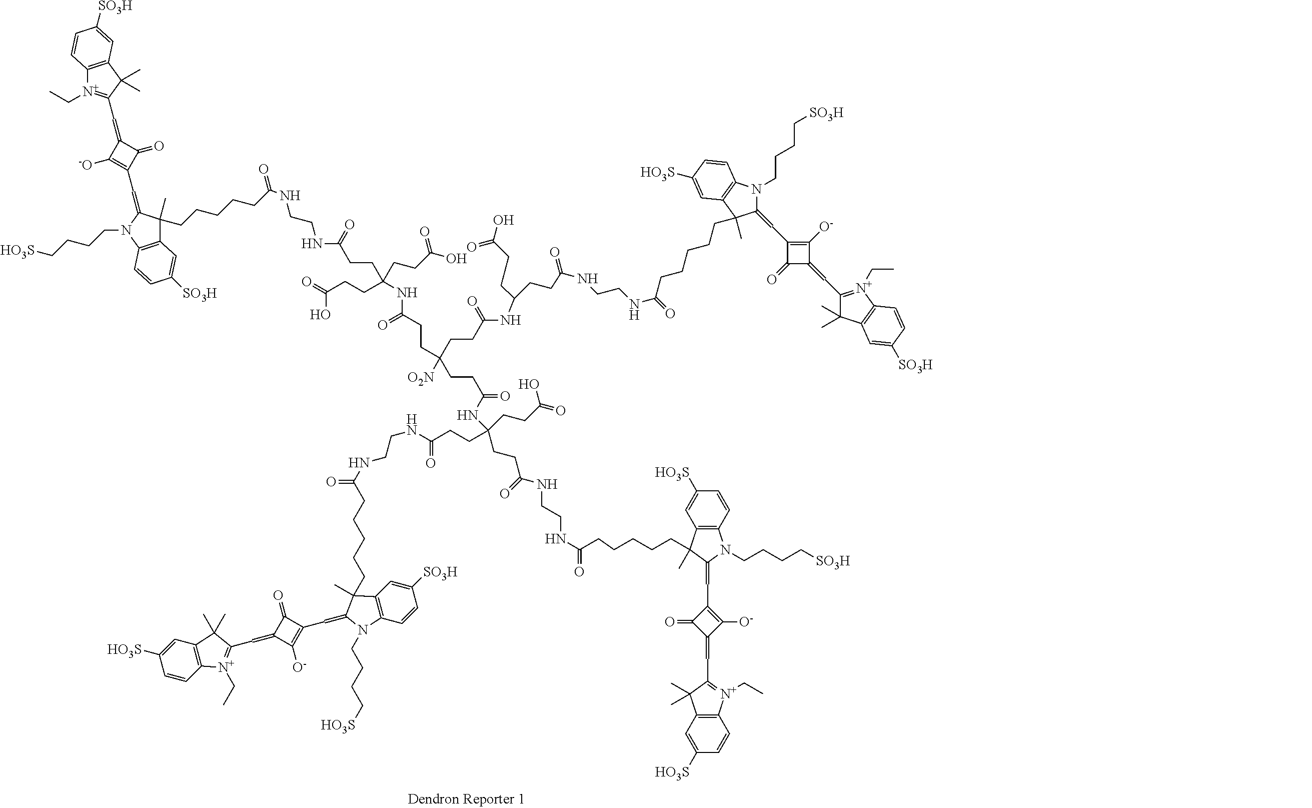

### Example 3

### Synthesis of Dendron Reporter 2 (DR2)

Behera's amine (commercially available from Frontier Scientific (catalog number NTN1963) is reacted in a first step with the acid chloride of 5-azidopentanoic acid. A solution of azidopentanoic acid chloride available from Aldrich (5 mmol), Behera's amine 1 (2.1 g, 5 mmol), and Et₃N (600 mg, 6 mmol) in dry benzene (25 mL) are stirred at 25° C. for 20 h. The mixture is washed sequentially with aqueous NaHCO₃ (10%), water, cold aqueous HCl (10%), and brine. The organic layer is then dried (Na₂C0₃), concentrated in vacuo to give a residue which is chromatographed (Si02), eluting it with CH₂Cl₂ to remove some byproducts and then with EtOAc to give compound D4 as a white solid.

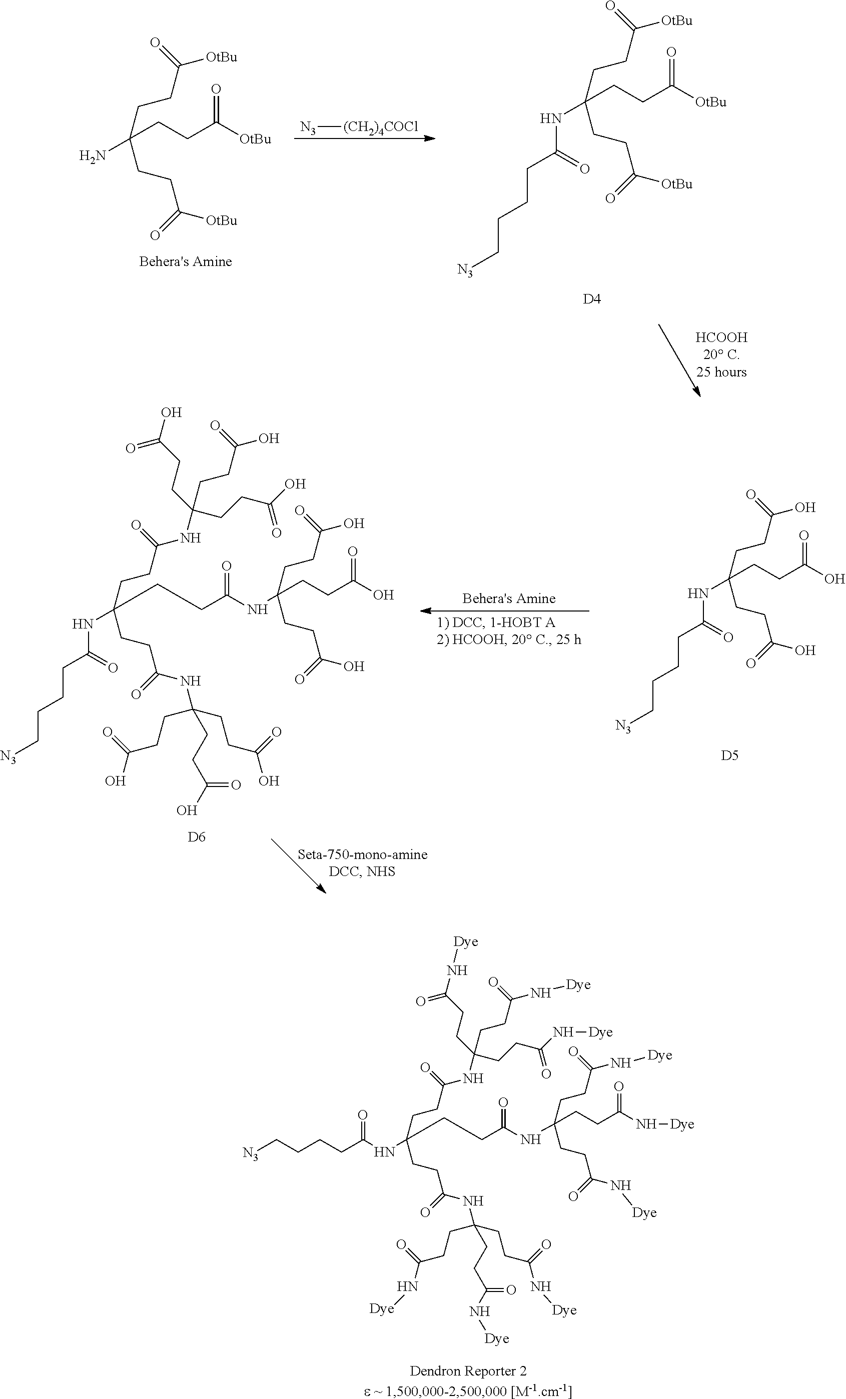

In a similar fashion other reactive, ionic and non-reactive groups can be introduced into Behera's amine. Acid chlorides and other functionalities capable of reaction with secondary amines can be used to introduce these groups. Some of these groups and cross-linkers are described in Greg Hermanson, Bioconjugate Techniques 2^{nd} Ed., Academic Press, Elsevier 2008 and are commercially available.

Hydrolysis of the t-butyl ester D4 [Newkome G. et al. (2003) Macromolecules 36, 4345-4354]:

A solution of the tert-butyl ester D4 (500 mmol) in formic acid (96%, 5 mL) is stirred at 25° C. for 25 h. The mixture is concentrated in vacuo to afford (100%) of the acid D5.

Synthesis of dendron structure D6 according to Newkome G. et al. (1991) J Org Chem. 56, 7162-7167:

A mixture of triacid D5 (1 mmol), Behera's amine (1.45 g, 3.5 mmol), dicyclohexylcarbodiimide (DCC; 620 mg, 3 mmol), and 1-hydroxybenzotrimle (400 mg, 3 mmol) in dry DMF (15 mL) is stirred at 25° C. for 48 h. After filtration of the dicyclohexylurea, the solvent is removed in vacuo. The residue is dissolved in CH₂Cl₂ (50 mL) and sequentially washed with cold aqueous HCl (10%), water, aqueous NaHCO₃, (10%), and brine. The organic phase is dried (Na₂SO₄). After removal of the solvent in vacuo the residue is subjected to flash chromatography (SiO₂, eluting with EtOAc and then 5% MeOH in EtOAc to yield the t-butyl ester of D6.

Hydrolysis of the t-butyl esters of D6 [Newkome G. et al. (2003) Macromolecules 36, 4345-4354]:

A solution of the tert-butyl ester of D6 (500 μmol) in formic acid (96%, 5 mL) is stirred at 25° C. for 25 h. The mixture was concentrated in vacuo to afford (100%) of the acid D6.

Labeling of the dendronic structure D6 with amino-modified acceptor dyes (e.g. Seta-750-amine):

The dendron D6 (1 μmol) containing 9 free carboxyl groups is dissolved in dry DMF. A 12-15-fold molar excess of amino-modified Seta-750 is added followed by a 15-fold excess of DCC and catalytic amounts of NHS. The solution is allowed to stir for about 10 hours; any precipitated urea is filtered; the solvent removed under reduced pressure and the residue is purified with reversed phase HPLC using an acetonitrile/water gradient to yield Dendron Reporter 2 (DR2).

Analogously any other amine-modified dye (see above) can be attached to the dendron. A series of amine-containing dyes are also listed in Richard P. Haugland, HANDBOOK OF FLUORESCENT PROBES AND RESEARCH CHEMICALS (6^{th} ed. 1996).

Click-chemistry reaction of DR2 with dibenzylcyclooctyn (DBCO)-modified DNA involves the following steps to react the reagent to DNA or other carrier molecules:

1. Prepare the DBCO-containing DNA sample in the reaction buffer.

2. Dissolve 1 mg of compound 5 in DMSO or DMF to make 10 mM solution.

3. Add 5 to a final concentration of 50-200 μM to the DNA sample. If the DNA concentration is >5 mg/ml, a 10-fold molar excess of the reagent, for samples <5 mg/ml, a 20-fold molar excess will be used.

4. Incubate the reaction at room temperature for 1 h.

5. Excess reagent will be removed from the conjugate using a desalting column or a dialysis cassette.

### Example 4

### Synthesis of Dendron Reporter 3 (DR3)

Modification of Carboxyl Dendron D2 with Aminopropyl Azide

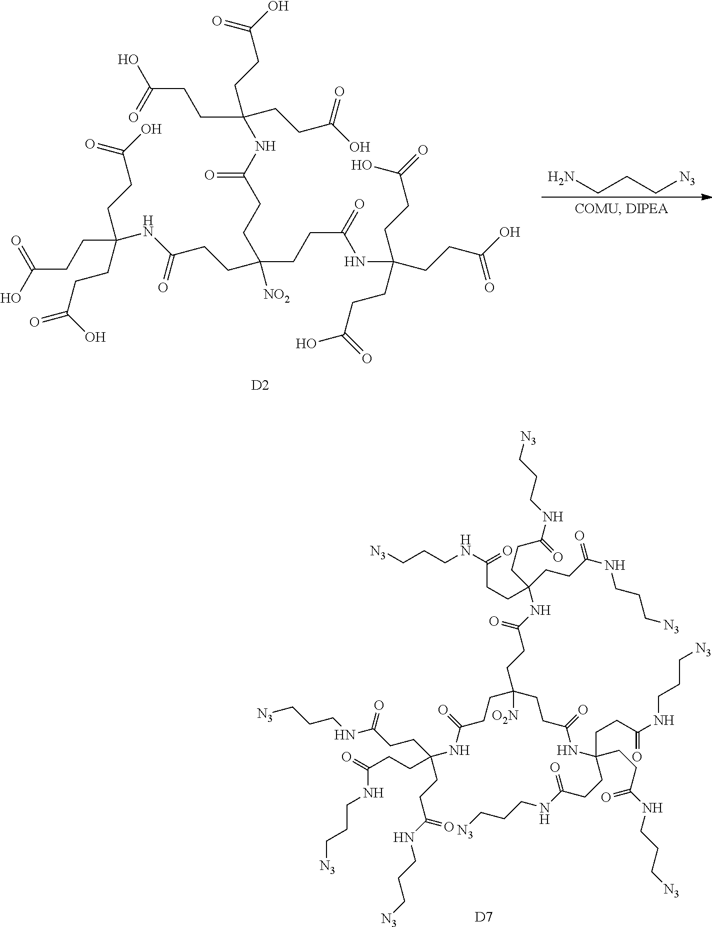 [SMILES: O=C(O)CCC(CCC(=O)O)(CCC(=O)O)NC(=O)CCC(CCC(=O)NC(CCC(=O)O)(CCC(=O)O)CCC(=O)O)(CCC(=O)NC(CCC(=O)O)(CCC(=O)O)CCC(=O)O)[N+](=O)[O-] ; [N-]=[N+]=NCCCN ; "D2" ; [N-]=[N+]=NCCCNC(=O)CCC(CCC(=O)NCCCN=[N+]=[N-])(CCC(=O)NCCCN=[N+]=[N-])NC(=O)CCC(CCC(=O)NC(CCC(=O)NCCCN=[N+]=[N-])(CCC(=O)NCCCN=[N+]=[N-])CCC(=O)NCCCN=[N+]=[N-])(CCC(=O)NC(CCC(=O)NCCCN=[N+]=[N-])(CCC(=O)NCCCN=[N+]=[N-])CCC(=O)NCCCN=[N+]=[N-])[N+](=O)[O-] ; "D7"]

86.8 mg (0.09 mmol) of hydrolyzed dendrimer D2 (containing nine free carboxylic groups) and 455 mg (1.06 mmol) of COMU were dissolved in 12 mL of dry DMF. 384 μL (2.2 mmol) of DIPEA and 162 mg (1.62 mmol) of aminopropyl azide were subsequently added and the mixture was stirred for 24 h at RT. The solvent was removed under reduced pressure and the residue was triturated with ether and dried in a vacuum desiccator. The product was column purified (Silica gel 0.063-0.1, methanol-ethyl acetate, 1:1).

¹H-NMR (200 MHz, DMSO-d₆), δ, ppm: 7.95 (9H, CONH, t, 4.9 Hz), 7.41 (3H, CONH, s), 3.34 (18H, CH₂N₃, t, 7.1 Hz), 3.07 (18H, CONHCH₂, q, 6.2 Hz, 12.2 Hz), 1.72-2.19 (48H, (CH₂)₂, m), 1.54-1.72 (18H, CH₂CH₂N₃, m).

Modification of Dye Sq2 with Propargyl Amine

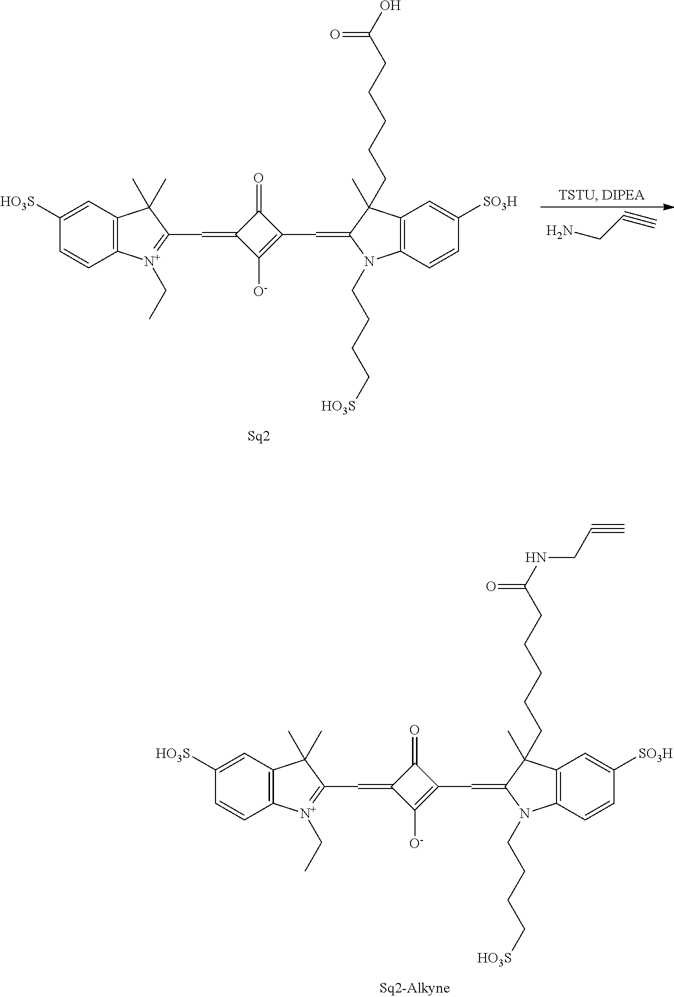

16 mg (0.02 mmol) of dye Sq2 and 12 mg (0.039 mmol) of TSTU were dissolved in 1 mL of dry DMF, 17 μL of DIPEA were added and the mixture was stirred for 1 h at RT. Then 2.2 mg (0.03 mmol) of propargyl amine and 17 μL of DIPEA were added to the obtained NHS ester and stirred 1 h at RT. The product was precipitated with ether, solvent was decanted and the residue was dried using a vacuum desiccator. The raw product was column purified (RP-18, 5-10% aq. acetonitrile) to give 6 mg of Sq2-Alkyne. UV-Vis: λₘₐₓ (abs) 633 nm (water); λₘₐₓ (em) 642 nm, quantum yield 6.5% (water).

¹H-NMR (200 MHz, DMSO-d₆), δ, ppm: 8.10-8.21 (3H, CONH and NH (DIPEA), m), 7.53-7.70 (4H, arom H, m), 7.19-7.34 (2H, arom H, m), 5.84 (1H, CH, s), 5.79 (1H, CH, s), 3.97-4.21 (4H, NCH₂, m), 3.69-3.78 (2H, NHCH₂CCH, m), 3.52-3.69 (4H, CH (DIPEA)), 3.04-3.21 (4H, CH₂ (DIPEA), m), 2.98 (1H, NHCH₂CCH, t, 2.5 Hz), 2.57-2.67 (2H, CH₂SO₃H, m), 1.87 (2H, CH₂CO, t, 7.1 Hz), 1.60-1.81 (4H, CH₂, m), 1.69 (3H, CH₃, s), 1.66 (9H, (CH₃)₂, s), 1.11-1.38 (37H, CH₃ (DIPEA) and CH₃ (ethyl), CH₂ (dye), m), 0.87-1.10 (2H, CH₂, m), 0.35-0.76 (2H, CH₂, m).

Click Chemistry Reaction Between Azide-Modified Dendrimer D7 and Sq2-Alkyne

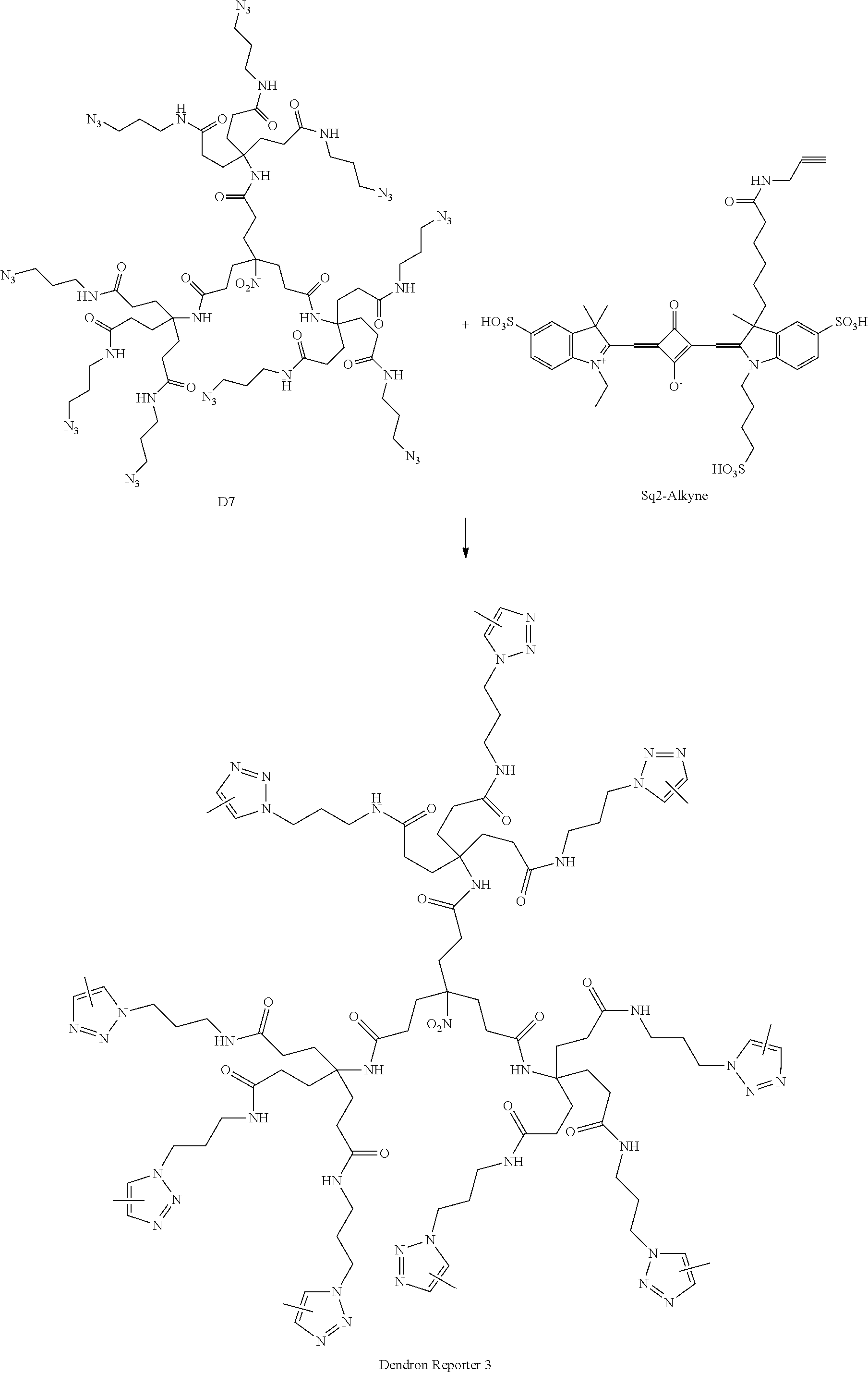

3 mg (0.00176 mmol) of azide-modified dendrimer D7 and 15.64 mg (0.0158 mmol) of Sq2-alkyne were dissolved in 1 mL of an ethanol-water (1:1) mixture. 15.8 μL of 0.1 M sodium ascorbate (0.00158 mmol) were added, purged with argon and 10 μL of a solution containing 3.94 mg of CuSO₄.5H₂O in 1 mL water (0.000158 mmol) were added. The mixture was stirred for 24 h at RT. The solvent was removed under reduced pressure; the residue was triturated with ether and dried using a vacuum desiccator. The product was column purified (RP-18, 5-8% aq. acetonitrile). UV-Vis: λₘₐₓ (abs) 633 nm (water); λₘₐₓ (em) 642 nm, quantum yield 6.3% (water).

¹H-NMR (200 MHz, DMSO-d₆), δ, ppm: 8.85 (9H, CH-azol, s), 8.10-8.47 (18H, NH (DIPEA), m), 7.85-8.02 (9H, CONH, m), 7.67-7.80 (9H, CONH, m), 7.54-7.68 (36H, arom, m), 7.41 (3H, CONH, s), 7.21-7.35 (18H, arom H, m), 5.84 (9H, CH, s), 5.79 (9H, CH, s), 3.94-4.25 (36H, NCH₂, m), 3.67-3.80 (18H, CONHCH₂), m), 3.51-3.67 (36H, CH (DIPEA)), 3.00-3.21 (72H, m), 2.57-2.68 (18H, CH₂SO₃H, m), 2.07-2.30 (24H, CH₂ dendr., m), 1.79-2.00 (78H, (CH₂)₂, m), 1.54-1.79 (54H, CH₂, m), 1.68 (27H, CH₃, s), 1.66 (36H, (CH₃)₂, s), 0.90-1.38 (351H, CH₃ (DIPEA) and CH₃ (ethyl), CH₂, m), 0.27-0.90 (18H, CH₂, m).

### Example 5

### Synthesis of Dendron Reporter 4 (DR4) and Dendron Reporter 5 (DR5)

Synthesis of Sq3-Amine-BOC

To a solution of 100 mg (0.15 mmol) of Sq3 and 70 mg (0.23 mmol) of TSTU in 2 mL of DMF 100 μL (0.574 mmol) of DIPEA were added and stirred for 1 h at RT. Then 32 mg (0.15 mmol) of N—BOC-1,6-hexanediamine and 100 μL of DIPEA were added and stirred at RT for 1 h. The product was precipitated with ether, solvent decanted and the residue was column purified (RP-18, 0-30% aq. acetonitrile). Yield 57.7 mg.

¹H-NMR (200 MHz, DMSO-d₆), δ, ppm: 13.30 (1H, NH, s), 7.99-8.40 (2H, NH (DIPEA), broad s), 7.72 (1H, CONH, s), 7.68 (1H, arom H, s), 7.65 (1H, arom H, s), 7.56 (2H, arom H, d, 8.3 Hz), 7.27 (1H, arom H, d, 8.2 Hz), 7.18 (1H, arom H, d, 8.1 Hz), 6.67-6.81 (1H, NH—BOC, broad s), 5.71 (1H, CH, s), 5.63 (1H, CH, s), 3.94-4.18 (2H, NCH₂, m), 3.50-3.74 (4H, CH of DIPEA, m), 3.06-3.19 (4H, CH₂ (ethyl) of DIPEA, m), 2.77-3.07 (4H, NHCH₂, m), 2.03 (2H, CH₂, t, 6.4 Hz), 1.62-1.78 (2H, CH₂, m), 1.66 (6H, CH₃, s), 1.45 (6H, CH₃, s), 1.41-1.60 (4H, CH₂, m), 1.35 (9H, OC(CH₃)₃, s), 1.14-1.32 (38H, m).

Removal of Protection Group from Sq3-Amine-BOC

41.7 mg (0.037 mmol) of Sq3-Amine-BOC were dissolved in 2.5 mL water, 400 μL of TFA were added and stirred at RT for 2 h. The solvent was removed using a rotary evaporator and the residue was triturated with ether to give 40 mg of raw Sq3-Amine which was used for following conjugation without additional purification.

1H NMR (200 MHz, DMSO-d6), δ, ppm: 13.32 (1H, NH (dye), s), 8.05-8.36 (2H, NH (DIPEA), m), 7.74 (1H, CONH, s), 7.69 (1H, arom H, s), 7.66 (1H, arom H, s), 7.55 (2H, arom H, d, 8.3 Hz), 7.27 (1H, arom H, d, 8.1 Hz), 7.17 (1H, arom H, d, 8.1 Hz), 5.72 (1H, CH, s), 5.64 (1H, CH, s), 4.02-4.17 (2H, NCH2, m), 3.05-3.22 (4H, CH2 (ethyl) of DIPEA, m), 2.88-3.04 (2H, NHCH2, m), 2.69-2.79 (2H, CH2NH2, m), 2.01 (2H, CH2, t, 6.4 Hz), 1.62-1.79 (2H, CH2, m), 1.66 (6H, CH3, s), 1.45 (6H, CH3, s), 1.38-1.60 (4H, CH2, m), 1.13-1.36 (38H, m).

Reaction of Sq3-Amine with Dendron D2

To a solution of 2 mg (0.00207 mmol) of dendron D2 and 10 mg (0.032 mmol) of TBTU in 1 mL of DMF 60 mg of Sq3-Amine and 33 μL (0.188 mmol) of DIPEA were added. The mixture was stirred at RT for 24 h, the solvent was removed using a rotary evaporator and the product was column purified (RP-18). The two main fraction containing dendron reporter compounds were collected, the first fraction was eluted with 20% aqueous acetonitrile contains five dye molecule on one dendrimer (DR4), the second was eluted with 35% of aqueous acetonitrile contains nine dye molecule on one dendrimer (DR5). The absorption and emission spectrum of DR4 is shown in FIG. 2.

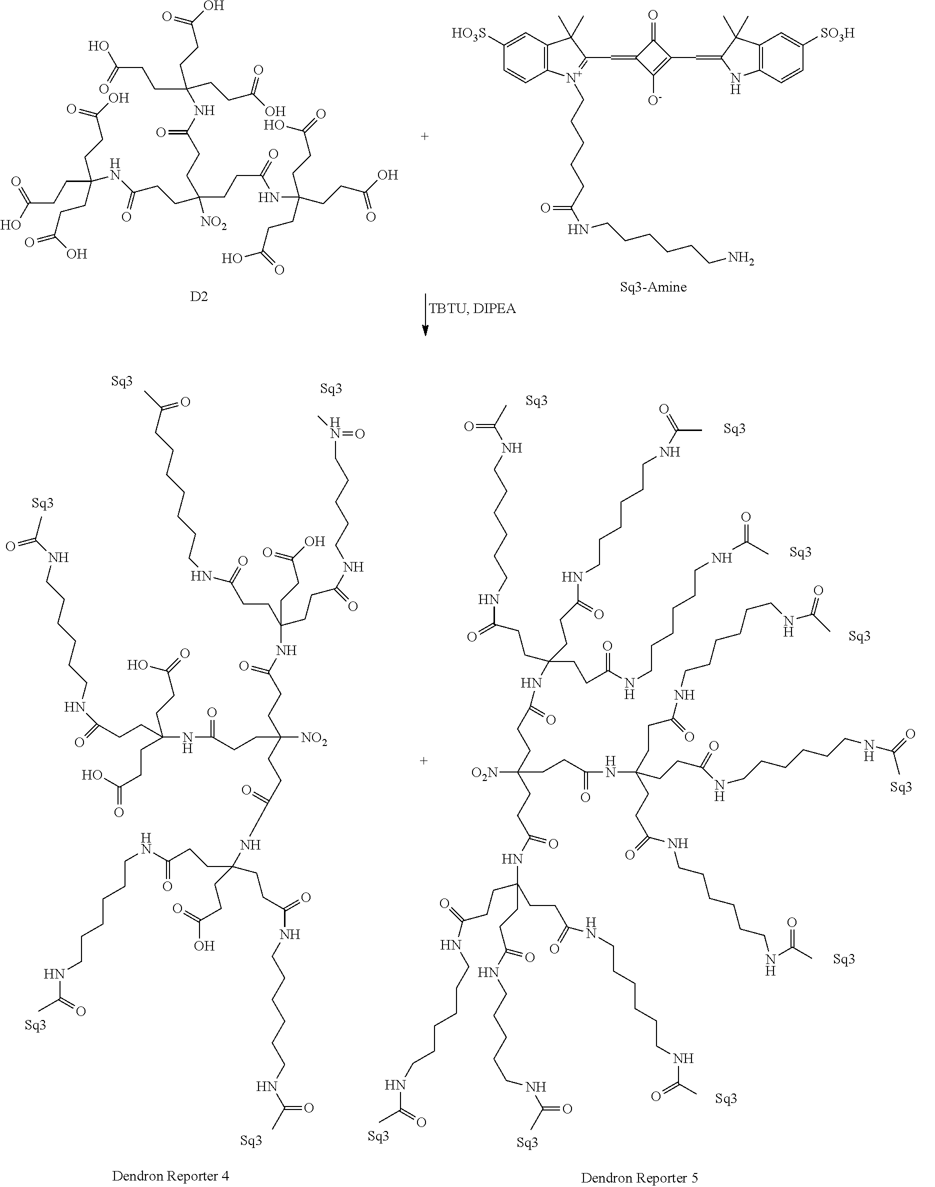

¹H-NMR (200 MHz, DMSO-d₆), δ, ppm for DR4: 13.25 (5H, NH (dye), s), 8.10-8.35 (5H, NH (DIPEA), m), 7.71-7.85 (10H, CONH, m), 7.47-7.71 (20H, arom H, m), 7.38 (3H, CONH, s), 7.28 (5H, arom H, d, 8.3 Hz), 7.19 (5H, arom H, d, 8.4 Hz), 5.71 (5H, CH, s), 5.63 (5H, CH, s), 3.90-4.18 (10H, NCH₂, m), 3.73-3.53 (10H, CH (isopropyl of DIPEA), m), 3.06-3.20 (10H, CH₂ (ethyl) of DIPEA, m), 2.85-3.03 (20H, NHCH₂, m), 1.84-2.17 (40H, CH₂, m), 1.70-1.84 (28H, CH₂, m), 1.66 (30H, CH₃, s), 1.43 (30H, CH₃, s), 1.49-1.58 (10H, CH₂ (dye), m), 1.29-1.38 (10H, CH₂ (dye), m), 1.12-1.29 (115H, CH₃ (DIPEA) and CH₂ (amine linker), m).

¹H-NMR (200 MHz, DMSO-d₆), δ, ppm for DR5: 13.27 (9H, NH (dye), s), 8.14-8.36 (9H, NH (DIPEA), m), 7.71-7.85 (18H, CONH, m), 7.47-7.71 (36H, arom H, m), 7.38 (3H, CONH, s), 7.28 (9H, arom H, d, 8.4 Hz), 7.19 (9H, arom H, d, 8.4 Hz), 5.70 (9H, CH, s), 5.63 (9H, CH, s), 3.90-4.18 (18H, NCH₂, m), 3.73-3.53 (18H, CH (isopropyl of DIPEA), m), 3.06-3.20 (18H, CH₂ (ethyl) of DIPEA, m), 2.88-3.03 (36H, NHCH₂, m), 1.84-2.17 (48H, CH₂, m), 1.70-1.84 (36H, CH₂, m), 1.66 (54H, CH₃, s), 1.43 (54H, CH₃, s), 1.49-1.58 (18H, CH₂ (dye), m), 1.29-1.38 (18H, CH₂ (dye), m), 1.12-1.29 (207H, CH₃ (DIPEA) and CH₂ (amine linker), m).

TABLE II
Spectral properties of the DR4, DR5 and Sq-3-Amine in
phosphate buffer pH 7.4

| | | Extinction | | |
|---|---|---|---|---|
| | Absorption | Coefficient | Emission | Quantum |
| Sample | max. [nm] | [M⁻¹ cm⁻¹] | max. [nm] | Yield [%] |
| Dye Sq-3-Amine | 635 | 200,000 | 647 | 8 |
| Dendron Reporter | 636 | Not measured | 651 | 12 |
| 4 | | | | |
| Dendron Reporter | 591, 639 | Not measured | 651 | 1 |
| 5 | | | | |

### Example 6

### Synthesis of the Dendron Reporter 6 (DR6)

Reaction of Dye-N1-Amine with Dendron D2:

To a solution of 1.9 mg (1.97 μmol) of dendron D2 and 7.2 mg (23.9 μmol) of O—(N-succinimidyl)-N,N,N′,N′-tetramethyluronium tetrafluoroborate (TSTU) were dissolved in 700 μL of DMF, then 9 μL (51.7 μmol) of N,N-diisopropyl ethyl amine (DIPEA) were added. The solution was stirred at room temperature for 1 hour and then solution of 30 mg (59.6 μmol) of N1-Amine in 1.5 mL of DMF and 20 μL (115 μmol) of DIPEA were successively added. The mixture was stirred at RT for 30 h, the solvent was removed using a rotary evaporator. Separation of the dendron reporter 6 (DR6) compound from the free dye was achieved using gel permeation chromatography on a 1.5×25 cm column with Sephadex G-15. The fraction with the shortest retention time contains the DR6.

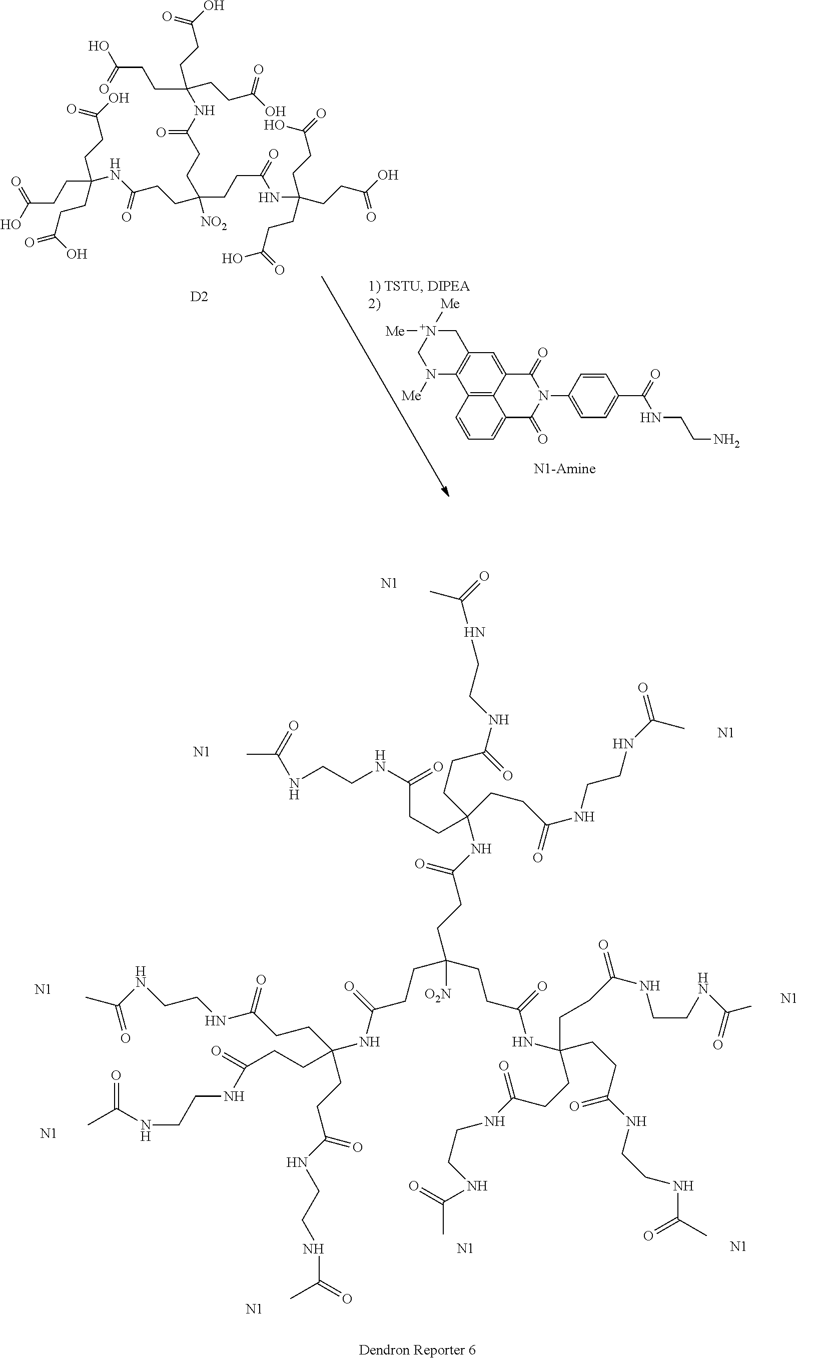

¹H-NMR (200 MHz, DMSO-d₆), δ, ppm: 8.66 (9H, arom H, d, 8.2 Hz), 8.55 (9H, arom H, d, 7.3 Hz), 8.24 (9H, arom H, s), 8.01 (18H, arom H, d, 7.6 Hz), 7.96 (9H, arom H, t, 8.1 Hz), 7.74-7.92 (21H, CONH, m), 7.49 (18H, arom H, d, 7.7 Hz), 5.04 (18H, CH₂, s), 4.90 (18H, CH₂, s), 3.67 (27H, 4-NCH₃, s), 3.21 (54H, ⁺N(CH₃)₂, s), 3.07 (36H, NCH₂CH₂N, broad s), 1.80-2.32 (48H, CH₂ (dendron), m).

TABLE III
Spectral properties of DR6 and Dye N1 in phosphate
buffer pH 7.4

| | | Extinction | |
|---|---|---|---|
| | Absorption | Coefficient | Emission |
| Sample | max. [nm] | [M⁻¹ cm⁻¹] | max. [nm] |
| Dye N1 | 405 | 13,800 | 518 |
| Dendron Reporter 6 | 408 | 95,000 | 520 |

### Example 7

### Labeling of DR1 to IgG

Protein labeling reactions were carried out using 67 mM phosphate buffer (pH 7.5). A stock solution of 1 mg of the NHS-activated DR1 in 100 μL of anhydrous DMF was prepared; 1 mg of IgG were dissolved in 0.5 mL of a 67 mM phosphate buffer (pH 7.5) and a series of labeling reactions with 5, 10, 20, and 50 μL of the dye stock solution were set up to obtain different dye-to-protein ratios (D/P) and the mixtures were allowed to stir overnight at room temperature.

Unconjugated dye was separated from the labeled proteins using gel permeation chromatography with Sephadex G25 (0.5 cm×20 cm column) and a 67 mM phosphate buffer solution of pH 7.4 as the eluent.

Calculation of the Dye-to-Protein Ratio

The dye-to-protein ratio (D/P) is calculated using the equation:

$$D / P = \frac{{A}_{conj ( λ max )} {ɛ}_{p}}{( {A}_{conj ( 278 )} - {xA}_{conj ( λ max )} ) {ɛ}_{dye}}$$

where A_{conj(λmax)}, A_{conj(278)} are the absorbances at absorption maxima and at 278 nm of the dye-protein conjugate respectively; ε_{dye} is the extinction coefficient of the dye at λₘₐₓ, for the DR1 is 725,000 M⁻¹cm⁻¹.

εₚ is the extinction coefficient of the protein at 278 nm, for IgG: εₚ=201,700 M⁻¹cm⁻¹.

The factor x in the denominator accounts for dye absorption at 278 nm (A_{dye(278)}) which is a percent of the absorption of the dye at its maximum absorption (A_{dye(λmax)}) (x=A_{dye(278)}/A_{dye(λmax)}). The x factor value for DR1 is 0.07

TABLE IV
Spectral properties of DR1 and DR1-IgG conjugate in
phosphate buffer pH 7.4 for a D/P of 1:

| | | Extinction | | |
|---|---|---|---|---|
| | Absorption | Coefficient | Emission | Quantum |
| Sample | max. [nm] | [M⁻¹ cm⁻¹] | max. [nm] | Yield [%] |
| DR1 | 633 | 725,000 | 642 | 9 |
| DR1-IgG conjugate | 638 | 725,000 | 648 | 20 |

Applications of the Invention

The dendron reporter compounds are useful as labels for various assay formats but in particular for FRET based assays and FRET based applications as donors and acceptors.

The assay may be a competitive assay that includes a recognition moiety, a binding partner, and an analyte. Binding partners and analytes may be selected from the group consisting of biomolecules, drugs, and polymers, among others. In some competitive assay formats, one or more components are labeled with photoluminescent compounds in accordance with the invention. For example, the binding partner may be labeled with such a photoluminescent compound, and the displacement of the compound from an immobilized recognition moiety may be detected by the measurement of luminescence coming from the reporter in the liquid phase of the assay.

The binding of antagonists to a receptor can be assayed by a competitive binding method in so-called ligand/receptor assays. In such assays, a labeled antagonist competes with an unlabeled ligand for the receptor binding site. One of the binding partners can be, but not necessarily has to be, immobilized. Such assays may also be performed in microplates. Immobilization can be achieved via covalent attachment to the well wall or to the surface of beads.

Other preferred assay formats are immunoassays. There are several such assay formats, including competitive binding assays, in which labeled and unlabeled antigens compete for the binding sites on the surface of an antibody. Typically there are incubation times required to provide sufficient time for equilibration. Such assays can be performed in heterogeneous or homogeneous formats.

Sandwich assays may use secondary antibodies and excess binding material may be removed from the analyte by a washing step.

Other types of reactions include binding between avidin and biotin, protein A and immunoglobulins, lectins and sugars (e.g., concanavalin A and glucose).

Certain dendron reporters of the invention are charged due to the presence sulfonic, phosphate, phosphonate, ammonium, and carboxylic acid groups. These compounds are impermeant to membranes of biological cells. In these cases treatments such as electroporation and shock osmosis can be used to introduce the dye into the cell. Alternatively, such reporters can be physically inserted into the cells by pressure microinjection, scrape loading etc.

Recent studies show that dendrimers can be internalized into cells by endocytosis. The additional functionalization of the reporter labeled dendrons with specific carriers and reactive groups should allow these systems to be utilized also as a valid alternative for drug and gene delivery.

The reporter compounds described here also may be used to sequence nucleic acids and peptides. For example, fluorescently-labeled oligonucleotides may be used to trace DNA fragments. Other applications of labeled DNA primers include fluorescence in-situ hybridization methods (FISH) and for single nucleotide polymorphism (SNIPS) applications, among others.

Multicolor labeling experiments may permit different biochemical parameters to be monitored simultaneously. For this purpose, two or more reporter compounds are introduced into the biological system to report simultaneously on different biochemical functions. The technique can be applied to fluorescence in-situ hybridization (FISH), DNA sequencing, fluorescence microscopy, and flow cytometry. One way to achieve multicolor analysis is to label biomolecules such as nucleotides, antibodies and proteins or DNA primers with different luminescent reporters having distinct luminescence properties. Luminophores with narrow emission bandwidths are preferred for multicolor labeling (multiplexing), because they have only a small overlap with other dyes and hence increase the number of dyes possible in a multicolor experiment. Importantly, the emission maxima have to be well separated from each other to allow sufficient resolution of the signal. A suitable multicolor triplet of dendron-reporters would include a heptacyanine analog of this invention, tricyanine analog of this invention, and a pentacyanine analog as described herein, among others.

Phosphoramidites are useful functionalities for the covalent attachment to oligos in automated oligonucleotide synthesizers. They are easily obtained by reacting hydroxyalkyl-modified alkyl groups (in our invention X is OH) of the invention with 2-cyanoethyl-tetraisopropyl-phosphorodiamidite and 1-H tetrazole in methylene chloride.

The simultaneous use of FISH (fluorescence in-situ hybridization) probes in combination with different fluorophores is useful for the detection of chromosomal translocations, for gene mapping on chromosomes, and for tumor diagnosis, to name only a few applications. In this approach each nucleic acid probe is labeled with a different dendronic reporter molecule showing distinct spectral properties and used in a multicolor, multisequence analysis approach.

These compositions can be combined with various labeling technologies as described in US2008299637A1.

In another approach the reporters of this invention might be used to directly stain or label a sample so that the sample can be identified and or quantitated. Such reporters might be added or labeled to a target analyte as a tracer. Such tracers may be used in photodynamic therapy where the labeled compound is irradiated with a light source and thus producing singlet oxygen that helps to destroy tumor cells and diseased tissue samples.

The reporter compounds of the invention can also be used for screening assays for a combinatorial library of compounds. The compounds can be screened for a number of characteristics, including their specificity and avidity for a particular recognition moiety.

Assays for screening a library of compounds are well known. A screening assay is used to determine compounds that bind to a target molecule, and thereby create a signal change which is generated by a labeled ligand bound to the target molecule. Such assays allow screening of compounds that act as agonists or antagonists of a receptor, or that disrupt a protein-protein interaction. It also can be used to detect hybridization or binding of DNA and/or RNA.

Other screening assays are based on compounds that affect the enzyme activity. For such purposes, quenched enzyme substrates of the invention could be used to trace the interaction with the substrate. In this approach, the cleavage of the fluorescent substrate leads to a change in the spectral properties such as the excitation and emission maxima, intensity and/or lifetime, which allows distinguishing between the free and the bound luminescent reporter.

The reporter compounds disclosed above may also be relevant to single molecule fluorescence microscopy (SMFM) where detection of single probe molecules depends on the availability of luminescent reporters with high fluorescence yield, high photostability, and long excitation wavelength.

The reporter compounds are also useful for use as biological stains. There may be limitations in some instances to the use of the above compounds as labels. For example, typically only a limited number of dyes may be attached to a biomolecules without altering the fluorescence properties of the dyes (e.g. quantum yields, lifetime, emission characteristics, etc.) and/or the biological activity of the bioconjugate. Compounds claimed here may be also used for covalent and non-covalent labeling of proteins and other biomolecules in gel-electrophoresis applications.

Compounds of this invention may also be attached to the surface of metallic nanoparticles such as gold or silver nanoparticles. It has recently been demonstrated that fluorescent molecules may show increased quantum yields near metallic nanostructures e.g., gold or silver nanoparticles (O. Kulakovich et al., Nanoletters 2 (12) 1449-52, 2002). This enhanced fluorescence may be attributable to the presence of a locally enhanced electromagnetic field around metal nanostructures. The changes in the photophysical properties of a fluorophore in the vicinity of the metal surface may be used to develop novel assays and sensors. In one example the nanoparticle may be labeled with one member of a specific binding pair (antibody, protein, receptor, etc.) and the complementary member (antigen, ligand) may be labeled with a fluorescent molecule in such a way that the interaction of both binding partners leads to an detectable change in one or more fluorescence properties (such as intensity, quantum yield, lifetime, among others). Replacement of the labeled binding partner from the metal surface may lead to a change in fluorescence that can then be used to detect and/or quantify an analyte.

Analytes

The reporters of this invention may be used to detect an analyte by introducing dye molecules as recognition moieties in these reporters. Such recognition moieties allow the detection of specific analytes. Calcium, potassium and pH sensing molecules are well known in the literature. Calcium-sensors based on the BAPTA (1,2-Bis(2-aminophenoxy)ethan-N,N,N′,N′-tetra-aceticacic) chelating moiety are frequently used to trace intracellular ion concentrations. The combination of a reference dye, relatively insensitive to the analyte, and a sensor dye, relatively sensitive to the analyte (e.g., a pH indicator which changes the emission spectrum with pH), on the dendronic backbone allows one to generate dendron reporters for ratiometric detection of pH. Analogously, ratiometric sensors for any other type of analyte could be generate by this principle. In addition, the flexibility of varying the focal-point X on the dendron backbone allows for reactive or non-reactive dendron reporter molecules, as required by the particular application.

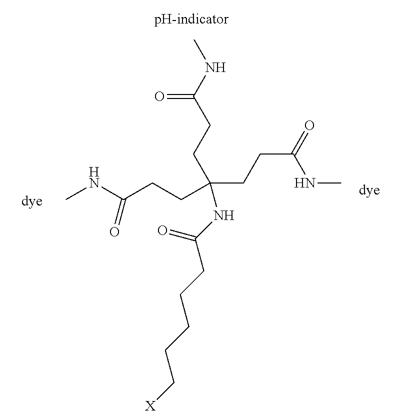

X=CO—NHS, SH, carboxyl, maleimide, iodoacetamide, phosphoramidite, isothiocyanate, alkyl, an ionic group or a linked carrier.

Fluorescence Methods

The disclosed reporter compounds may be detected using common intensity-based fluorescence methods but they are in particular interesting for FRET type applications as these dendronic reporters offer the possibility to pack the highest possible number of dyes within the smallest volume element possible, allowing the generation of fluorescent resonance energy transfer (FRET) donors and acceptors that are capable of expanding the measurable range of FRET to beyond the current limit of around 80 Å.

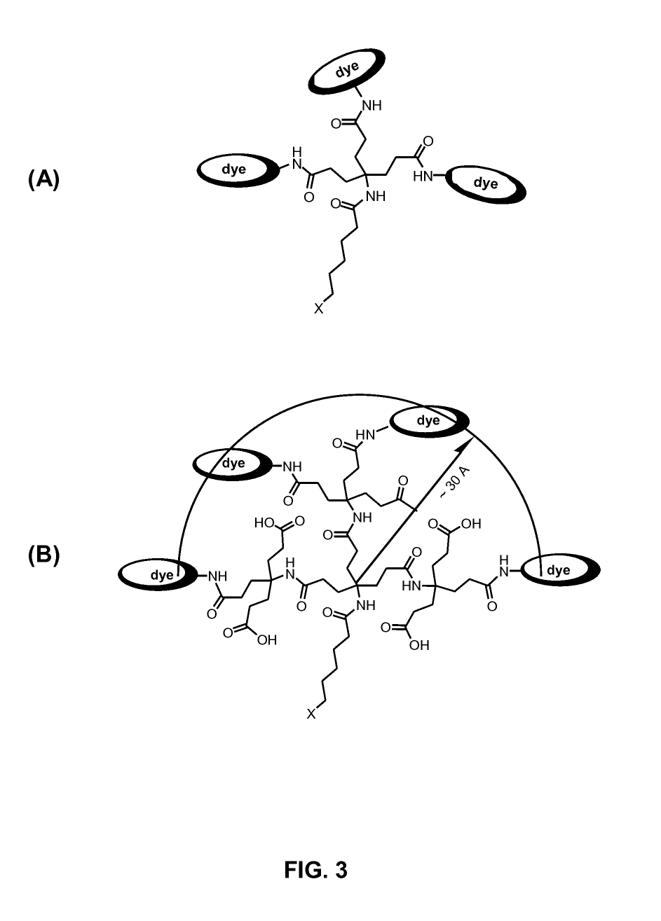

It has already been demonstrated by measuring the quantum yield of DR1 that the labeling of 4 Sq1-dyes on the dendron backbone in very close proximity to each other (˜20-40 Å, as shown in FIG. 3b, Generation 2) does not lead to quenching of the dye fluorescence as is the case when the same dyes are labeled to IgG, a protein much larger in size (F_{ab} fragment ˜60 Å and the length of an IgG is around 115 Å, Journal of Virology 77(19), 10557-10565, 2003) or bovine serum albumin (BSA), a protein of similar dimensions (BSA is postulated to be an oblate ellipsoid with dimensions of 140×40×40 Å; Bendedouch, D., and Chen, S. H., J. Phys. Chem. 87; 1473-1477, 1983) but rather in an increase in quantum yield by approximately 50%. On the other hand, upon increasing the dye to protein ratio from 1 to 4 Sq1-dyes per protein on IgG or BSA, the quantum yield decreases by 56% and 65% respectively.

The dendron reporter molecules are also suitable for a lifetime-based read-out option. Preferred assays with fluorescence lifetime as a read-out parameter include for example FRET assays. The binding between a fluorescent donor labeled species (typically an antigen) and a fluorescent acceptor labeled species may be accompanied by a change in the intensity and the fluorescence lifetime. The lifetime can be measured using time-correlated-single-photon-counting (TSPC) or phase-modulation-based methods (J. R. LAKOWICZ, PRINCIPLES OF FLUORESCENCE SPECTROSCOPY (2^{nd} Ed. 1999)).

Moreover, internal lifetime systems may be generated by combining an analyte-sensitive reporter molecule (R¹) that is non-fluorescent but changes its spectral overlap with the emission of a luminescent reference molecule (R²) on the dendron backbone. It is understood that the Förster distances in this internal FRET based reporters can be controlled by using different generation dendron backbones (e.g. 2^{nd}, 3^{rd}, 4^{th}, or 5^{th} generation).

These new compositions are also useful as tracers in fluorescence polarization (FP) assays. Fluorescence polarization immunoassays (FPI) are widely applied to quantify low molecular weight antigens. The assays are based on polarization measurements of antigens labeled with fluorescent probes. The requirement for polarization probes used in FPIs is that emission from the unbound labeled antigen be depolarized and increase upon binding to the antibody. Low molecular weight species labeled with the compounds of the invention may be used in such binding assays, and the unknown analyte concentration may be determined by the change in polarized emission from the fluorescent tracer molecule.

An important aspect of these dendron reporters for polarization measurements is that they allow the adjustment of the luminescent lifetime of the dendron polarization label by internal energy transfer, thereby enabling one to optimize the sensitivity of a polarization based assay.

Due to the multi-component nature of these dendron reporters, compositions of the invention are expected to have high two-photon cross sections for use in two-photon applications where the reporter is excited with wavelengths in the NIR region from 700-1000 nm, typically using a Ti-Sapphire laser system. Dendrimer-based two-photon reporters with very high cross sections are claimed in PCT Appl. WO 2007/080176.

The compositions are useful for single molecule measurements due to the fact that the presence of several dye molecules in the dendron reporter will help to increase the measurement time of the labeled species under the microscope before total bleaching.

Compositions and Kits

The invention also provides compositions, kits and integrated systems for practicing the various aspects and embodiments of the invention, including producing the novel compounds and practicing of assays. Such kits and systems may include a reporter compound as described above, and may optionally include one or more of solvents, buffers, calibration standards, enzymes, enzyme substrates, and additional reporter compounds having similar or distinctly different optical properties.

It should be emphasized that the above-described embodiments of the invention are merely possible examples of implementations of the invention. Many variations and modifications may be made to the above-described embodiments. All such modifications and variations are intended to be included herein within the scope of this disclosure and protected by the following claims.

1. A dendron reporter comprising
covalently linked dendron units, DU, of the formula 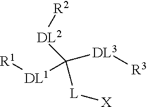
wherein L, DL¹, DL², and DL³ are each independently a single covalent bond or a bivalent linkage that is linear, cyclic or heterocyclic, saturated or unsaturated, having 1-20 non-hydrogen atoms from the group of C, N, P, O, and S, in such a way that the linkage contains any combination of ether, thioether, amine, ester, amide bonds; single, double, triple or aromatic carbon-carbon bonds; or carbon-sulfur bonds, carbon-nitrogen bonds, phosphorus-sulfur, nitrogen-nitrogen, nitrogen-oxygen, or aromatic or heteroaromatic bonds;
wherein periphery R¹, R², and R³ substituents are each independently H, alkyl, aryl, alkyl-aryl, amino, amido, ether, hydroxyl, thiol, substituted thiol, carboxyl, carboxylic ester, substituted amino, sulfo, phosphate, phosphonate groups, or a linked dye, provided that at least two of the periphery R¹, R², R³ substituents are linked dyes;
wherein non-periphery R¹, R², R³ substituents are X of another dendron unit DU; and
wherein the focal-point substituent X is a reactive group, ionic group or a conjugated substance.

2-4. (canceled)

5. The dendron reporter of claim 1 wherein the dendron reporter has a mass to dye ratio of 2,500 g M⁻¹ or less.

6. (canceled)

7. The dendron reporter of claim 1 wherein the focal-point substituent X is the only reactive group.

8. The dendron reporter of claim 1 wherein all DL¹, DL², and DL³ that link periphery R¹, R², R³ substituents that are linked dyes include a triazole group.

9. The dendron reporter of claim 1 wherein L has the formula 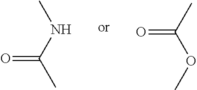
and wherein non-periphery DL¹, DL², and DL³ are each independently linear chains having 1-3 carbon atoms, carboxylic acid or carboxylic ester groups.

10. The dendron reporter of claim 1 wherein all dyes are identical.

11. The dendron reporter of claim 10 wherein the dye is selected from the group consisting of a squaraine dye, a cyanine dye, a squaraine rotaxane dye, a styryl dye, an oxazine dye, a polyaromatic dye, a naphthalic acid derivative, a perylenetetracarboxylic acid derivative, an oxazole derivative, an oxadiazole derivative, a heterocyclic dye, a xanthene dye, a rhodamine dye, a BODIPY dye, a coumarin dye, a phthalocyanine dye, a porphyrine dye, a Ru-, Os- or Re-metal-ligand complex, and a lanthanide complex.

12-19. (canceled)

20. The dendron reporter of claim 1 having the formula: 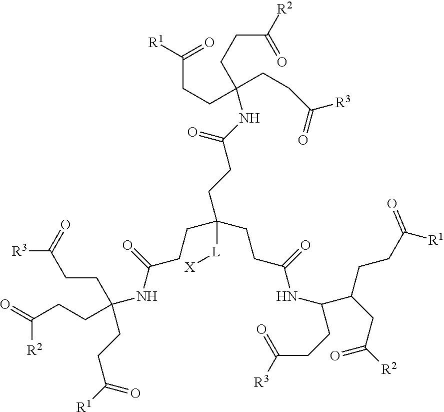
wherein R¹, R², and R³ are each independently hydroxy, alkoxy, amino, a substituted amino group, or a linked dye; L is a linker selected from the group of —NH—CO—(CH₂)ₙ—, —O—(CH₂)ₙ—, —(O—CH₂—CH₂)ₙ—, and —S—(CH₂)ₙ—, where n is 1 to 10;
wherein at least one R¹, at least one R², or at least one R³ includes a first dye and at least one other of R¹, R², or R³ includes a second dye.

21. The dendron reporter of claim 20 wherein the first dye and the second dye are identical.

22-23. (canceled)

24. The dendron reporter of claim 20 wherein the first dye and the second dye independently are selected from the following dye structures: 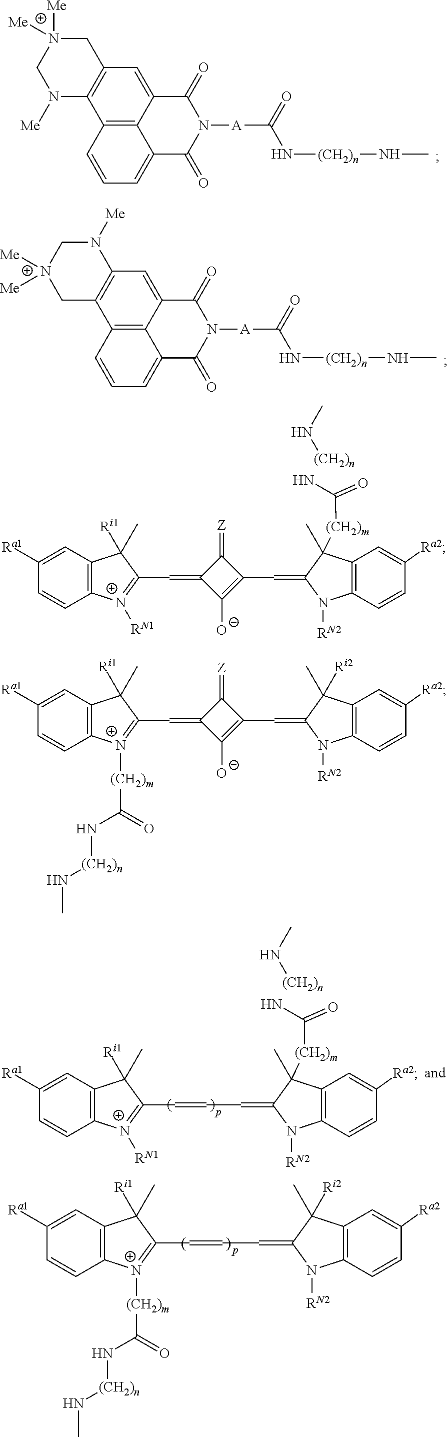
where:
Z=O, S, C(CN)₂;
R^{a1} and R^{a2} are independently H, SO₃H, COOH or carboxyalkyl group;
R^{N1} and R^{N2} are independently H, —(CH₂)ₖ—COOH, —(CH₂)ₖ—SO₃H, —(CH₂)ₖ—PO(OH)₃, —(CH₂)ₖ—PO(OAlk)₃, or an alkyl or alky-aryl group;
Rⁱ¹ and Rⁱ² are independently —(CH₂)ₖ—COOH, —(CH₂)ₖ—SO₃H, —(CH₂)ₖ—PO(OH)₃, —(CH₂)ₖ—PO(OAlk)₃, or an alkyl or alky-aryl group;
A=1,2-phenylene, 1,2-phenylene, 1,4-phenylene, or an aliphatic group; and
n=1-20; m=1-20; k=1-20; p=0-3.

25. The dendron reporter of claim 1 having the formula: 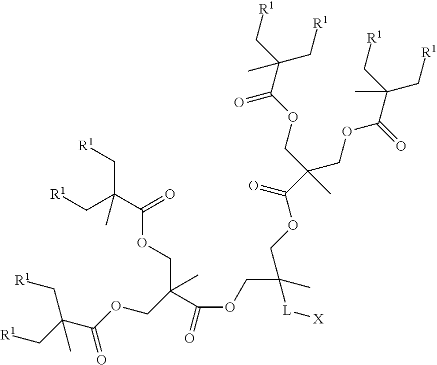
wherein R¹ are each independently hydroxy, alkoxy, carboxylic ester, a substituted carboxylic ester group, or a linked dye; L is carboxy or a carboxylic ester.

26. The dendron reporter of claim 25 wherein at least one of the dyes is different than another of the dyes.

27-28. (canceled)

29. The dendron reporter of claim 1 wherein at least one third of the periphery R¹, R², R³ substituents are linked dyes.

30. The dendron reporter of claim 29 wherein at least one half of the periphery R¹, R², R³ substituents are linked dyes.

31. The dendron reporter of claim 30 wherein substantially all periphery R¹, R², R³ substituents are linked dyes.

32. The dendron reporter of claim 1 wherein the generation is two or higher; and wherein at least one third of the periphery R¹, R², R³ substituents include an identical dye.

33. The dendron reporter of claim 32 wherein at least one of the periphery R¹, R², R³ substituents includes a dye different than the identical dye.

34. The dendron reporter of claim 1 wherein at least one of the periphery R¹, R², R³ substituents includes a first dye and at least one other of the periphery R¹, R², R³ substituents includes a second dye, and wherein the first dye and the second dye are different.

35. The dendron reporter of claim 34 wherein one of the first dye and the second dye is an energy transfer donor and the other of the first dye and the second dye is an energy transfer acceptor.

36. The dendron reporter of claim 34 wherein one of the first dye and the second dye changes photophysical properties in the presence of an analyte and the other of the first dye and the second dye is insensitive to the presence of the analyte.

37. The dendron reporter of claim 34 wherein one of the first dye and the second dye changes photophysical properties in relation to the concentration of an analyte and the other of the first dye and the second dye is insensitive to the concentration of the analyte.

38. A method of labelling a substance S^{c}, the method comprising:
linking at least four dyes to the substance S^{c} via a dendron that includes a dendron backbone, a focal point, and periphery sites;
wherein the dyes are linked to periphery sites of the dendron and the dendron is linked to the substance S^{c} at the focal point.

39. The method of claim 38 further comprising selecting the substance from the group consisting of an amino acid, a peptide, a protein, a nucleoside, a nucleotide, a nucleic acid, a carbohydrate, a lipid, an ion-chelator, a non-biological polymer, a cell, a cellular component, and a nanoparticle.
